SEQUENCE LISTING (51 sequences)

SEQ ID NO: 1 - protein, 504 aa, Artificial sequence
  Synthetic peptide
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 2 - protein, 448 aa, Oryctolagus cuniculus
QAQLEESGGDLVKPEGSLTLTCTASGFSFTDRHYMCWIRQAPGKGLEWIACVYPGSSGSTYYASWARGRFTISKTSSTTVTLRMTSLTAADTATYFCARSSYPDSSGYSYGMDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 3 - protein, 125 aa, Artificial sequence
  Synthetic peptide
QAQLEESGGDLVKPEGSLTLTCTASGFSFTDRHYMCWIRQAPGKGLEWIACVYPGSSGSTYYASWARGRFTISKTSSTTVTLRMTSLTAADTATYFCARSSYPDSSGYSYGMDLWGPGTLVTVSS

SEQ ID NO: 4 - protein, 9 aa, Artificial sequence
  Synthetic peptide
GFSFTDRHY

SEQ ID NO: 5 - protein, 8 aa, Artificial sequence
  Synthetic peptide
YPGSSGST

SEQ ID NO: 6 - protein, 17 aa, Artificial sequence
  Synthetic peptide
ARSSYPDSSGYSYGMDL

SEQ ID NO: 7 - protein, 214 aa, Oryctolagus cuniculus
DIVMTQTPASVEAAVGGTVTIKCQASQNINSNLAWYQQKPGQRPKFLMYLTSKLASGVPSRFRGSGSGTQFTLTISDLECADAATYYCQTYYDISNYGYAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 8 - protein, 110 aa, Artificial sequence
  Synthetic peptide
DIVMTQTPASVEAAVGGTVTIKCQASQNINSNLAWYQQKPGQRPKFLMYLTSKLASGVPSRFRGSGSGTQFTLTISDLECADAATYYCQTYYDISNYGYAFGGGTEVVVK

SEQ ID NO: 9 - protein, 6 aa, Artificial sequence
  Synthetic peptide
QNINSN

SEQ ID NO: 10 - protein, 3 aa, Artificial sequence
  Synthetic peptide
LTS

SEQ ID NO: 11 - protein, 12 aa, Artificial sequence
  Synthetic peptide
QTYYDISNYGYA

SEQ ID NO: 12 - DNA, 1404 bp, Oryctolagus cuniculus
atggagactgggctgcgctggcttctcctggtcgctgtgctcaaaggtgtccagtgtcaggcgcagctggaggagtccgggggagacctggtcaagcctgagggatccctgacactcacctgcacagcctctggattctccttcactgacaggcattacatgtgctggatccgccaggctccggggaaggggctggagtggatcgcatgcgtttatcctggtagtagtggtagcacttactacgcgagctgggcgagaggccgattcaccatctccaaaacctcgtcgaccacggtgactctgcgcatgaccagtctgacagccgcggacacggccacctatttctgtgcgagatcctcatatcctgatagtagtggctatagttatggcatggacctctggggccctgggaccctcgtcaccgtctcttcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 13 - DNA, 432 bp, Artificial sequence
  Synthetic nucleotide
atggagactgggctgcgctggcttctcctggtcgctgtgctcaaaggtgtccagtgtcaggcgcagctggaggagtccgggggagacctggtcaagcctgagggatccctgacactcacctgcacagcctctggattctccttcactgacaggcattacatgtgctggatccgccaggctccggggaaggggctggagtggatcgcatgcgtttatcctggtagtagtggtagcacttactacgcgagctgggcgagaggccgattcaccatctccaaaacctcgtcgaccacggtgactctgcgcatgaccagtctgacagccgcggacacggccacctatttctgtgcgagatcctcatatcctgatagtagtggctatagttatggcatggacctctggggccctgggaccctcgtcaccgtctcttca

SEQ ID NO: 14 - DNA, 714 bp, Oryctolagus cuniculus
atggacacgagggcccccactcagctgctggggctcctgttgctctggctcccaggtgccagatgtgctgacattgtgatgacccagactccagcctccgtggaggcagctgtgggaggcacagtcaccatcaagtgccaggccagtcagaacattaacagcaatttagcctggtatcagcagaaaccagggcagcgtcccaagttcctgatgtatctgacatccaaactggcatctggggtcccatcgcggttcagaggcagtggatctgggacacagttcactctcaccatcagcgacctggagtgtgccgatgctgccacttactactgtcaaacctattatgatattagtaattatggatatgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 15 - DNA, 399 bp, Artificial sequence
  Synthetic nucleotide
atggacacgagggcccccactcagctgctggggctcctgttgctctggctcccaggtgccagatgtgctgacattgtgatgacccagactccagcctccgtggaggcagctgtgggaggcacagtcaccatcaagtgccaggccagtcagaacattaacagcaatttagcctggtatcagcagaaaccagggcagcgtcccaagttcctgatgtatctgacatccaaactggcatctggggtcccatcgcggttcagaggcagtggatctgggacacagttcactctcaccatcagcgacctggagtgtgccgatgctgccacttactactgtcaaacctattatgatattagtaattatggatatgctttcggcggagggaccgaggtggtggtcaaa

SEQ ID NO: 16 - protein, 439 aa, Oryctolagus cuniculus
QSLEESGGDLVKPGASLTLTCTASGFDFSDRYYMCWVRQAPGKGLEWIGCIYVGSGDTYYASWAKGRFTISRTSSTTVTLQMTRLTAADTATYFCARHPGTYFTLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 17 - protein, 116 aa, Artificial sequence
  Synthetic peptide
QSLEESGGDLVKPGASLTLTCTASGFDFSDRYYMCWVRQAPGKGLEWIGCIYVGSGDTYYASWAKGRFTISRTSSTTVTLQMTRLTAADTATYFCARHPGTYFTLWGPGTLVTVSS

SEQ ID NO: 18 - protein, 9 aa, Artificial sequence
  Synthetic peptide
GFDFSDRYY

SEQ ID NO: 19 - protein, 7 aa, Artificial sequence
  Synthetic peptide
YVGSGDT

SEQ ID NO: 20 - protein, 8 aa, Artificial sequence
  Synthetic peptide
ARHPGTYF

SEQ ID NO: 21 - protein, 214 aa, Oryctolagus cuniculus
DVVMTQTPSSVSAAVGGTVTINCQASQNIVNNLAWYQQRPGQPPKLLIYDTSTLASGVPSRFKGSGSGTEFTLTISDLECADAATYYCQTYYYYNKIINGFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 22 - protein, 110 aa, Artificial sequence
  Synthetic peptide
DVVMTQTPSSVSAAVGGTVTINCQASQNIVNNLAWYQQRPGQPPKLLIYDTSTLASGVPSRFKGSGSGTEFTLTISDLECADAATYYCQTYYYYNKIINGFGGGTEVVVK

SEQ ID NO: 23 - protein, 6 aa, Artificial sequence
  Synthetic peptide
QNIVNN

SEQ ID NO: 24 - protein, 3 aa, Artificial sequence
  Synthetic peptide
DTS

SEQ ID NO: 25 - protein, 12 aa, Artificial sequence
  Synthetic peptide
QTYYYYNKIING

SEQ ID NO: 26 - DNA, 1377 bp, Oryctolagus cuniculus
atggagactgggctgcgctggcttctcctggtcgctgtgctcaaaggtgtccagtgtcagtcgttggaggagtccgggggagacctggtcaagcctggggcatccctgacactcacctgcacagcctctggattcgacttcagtgacagatattacatgtgttgggtccgccaggctccagggaaggggctggagtggatcggatgcatttatgttggtagtggtgacacttattatgcgagctgggcgaaaggccgattcaccatctccagaacctcgtcgaccacggtgactctgcaaatgacccgtctgacagccgcggacacggccacctatttctgtgcgagacatcctggtacttactttactttatggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 27 - DNA, 405 bp, Artificial sequence
  Synthetic nucleotide
atggagactgggctgcgctggcttctcctggtcgctgtgctcaaaggtgtccagtgtcagtcgttggaggagtccgggggagacctggtcaagcctggggcatccctgacactcacctgcacagcctctggattcgacttcagtgacagatattacatgtgttgggtccgccaggctccagggaaggggctggagtggatcggatgcatttatgttggtagtggtgacacttattatgcgagctgggcgaaaggccgattcaccatctccagaacctcgtcgaccacggtgactctgcaaatgacccgtctgacagccgcggacacggccacctatttctgtgcgagacatcctggtacttactttactttatggggcccaggcaccctggtcaccgtctcctca

SEQ ID NO: 28 - DNA, 711 bp, Oryctolagus cuniculus
atggacacgagggcccccactcagctgctggggctcctgctgctctggctcccaggtgccagatgtgatgttgtgatgacccagactccatcctccgtgtctgcagctgtgggaggcacagtcaccatcaattgccaggccagtcagaacattgtcaacaatttagcctggtatcagcagagaccagggcagcctcccaaactcctgatctatgatacatccactctggcatctggggtcccatcgcggttcaaaggcagtggatctgggacagagttcactctcaccatcagcgacctggagtgtgccgatgctgccacttactactgtcaaacctattattattataataaaattataaatggtttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 29 - DNA, 396 bp, Artificial sequence
  Synthetic nucleotide
atggacacgagggcccccactcagctgctggggctcctgctgctctggctcccaggtgccagatgtgatgttgtgatgacccagactccatcctccgtgtctgcagctgtgggaggcacagtcaccatcaattgccaggccagtcagaacattgtcaacaatttagcctggtatcagcagagaccagggcagcctcccaaactcctgatctatgatacatccactctggcatctggggtcccatcgcggttcaaaggcagtggatctgggacagagttcactctcaccatcagcgacctggagtgtgccgatgctgccacttactactgtcaaacctattattattataataaaattataaatggtttcggcggagggaccgaggtggtggtcaaa

SEQ ID NO: 30 - protein, 468 aa, Mus musculus
MRWSWIFLFLLSGTAGVLSEVQLQQSGPELVKPGTSVKISCKTSGYTFTEYTIHWVKQSHGKSLAWIGGIDPNSGGTNYSPNFKGKATLTVDKSSSTAYMDLRSLSSEDSAVYFCARIYHYDGYFDVWGAGTAVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 31 - protein, 119 aa, Artificial sequence
  Synthetic peptide
EVQLQQSGPELVKPGTSVKISCKTSGYTFTEYTIHWVKQSHGKSLAWIGGIDPNSGGTNYSPNFKGKATLTVDKSSSTAYMDLRSLSSEDSAVYFCARIYHYDGYFDVWGAGTAVTVSS

SEQ ID NO: 32 - protein, 8 aa, Artificial sequence
  Synthetic peptide
GYTFTEYT

SEQ ID NO: 33 - protein, 8 aa, Artificial sequence
  Synthetic peptide
IDPNSGGT

SEQ ID NO: 34 - protein, 8 aa, Artificial sequence
  Synthetic peptide
ARIYHYDG

SEQ ID NO: 35 - protein, 236 aa, Mus musculus
MDMRTPAQFLGILLLWFPGIKCDIKMTQSPSSMYASLGERVTITCKASQDINSYLTWFQQKPGKSPKTLIYRANRLIDGVPSRFSGSGSGQDYSLTISSLDYEDMGIYYCLQYDEFPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 36 - protein, 107 aa, Artificial sequence
  Synthetic peptide
DIKMTQSPSSMYASLGERVTITCKASQDINSYLTWFQQKPGKSPKTLIYRANRLIDGVPSRFSGSGSGQDYSLTISSLDYEDMGIYYCLQYDEFPPTFGGGTKLEIK

SEQ ID NO: 37 - protein, 6 aa, Artificial sequence
  Synthetic peptide
QDINSY

SEQ ID NO: 38 - protein, 3 aa, Artificial sequence
  Synthetic peptide
RAN

SEQ ID NO: 39 - protein, 8 aa, Artificial sequence
  Synthetic peptide
LQYDEFPP

SEQ ID NO: 40 - DNA, 1407 bp, Mus musculus
atgagatggagctggatctttctctttctcctgtcaggaactgcaggtgtcctctctgaggtccagctgcaacagtctggacctgagctggtgaagcctgggacttcagtgaagatatcctgcaagacttctggatacacattcactgaatataccatacactgggtgaagcagagccatggaaagagccttgcgtggattggaggtattgatcctaacagtggtggtactaactacagcccgaacttcaagggcaaggccacattgactgttgacaagtcctccagcacagcctacatggacctccgcagcctgtcatctgaggattctgcagtctacttctgtgcaaggatctatcattacgacggatacttcgatgtctggggcgcagggaccgcggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacagacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 41 - DNA, 711 bp, Mus musculus
atggacatgaggacccctgctcagtttcttggaatcttgttgctctggtttccaggtatcaaatgtgacatcaagatgacccagtctccatcttcaatgtatgcatctctaggagagagagtcactatcacttgcaaggcgagtcaggacattaatagctatttaacctggttccagcagaaaccagggaaatctcccaagaccctgatctatcgtgcaaacagattgatagatggggtcccatcaaggttcagtggcagtggatctgggcaagattattctctcaccatcagcagcctggactatgaagatatgggaatttattattgtctacagtatgatgagtttcctcccacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 42 - protein, 478 aa, Homo sapiens
MYRMQLLSCIALSLALVTNSEVQLVESGAEVKKPGASVKVSCKASGYTFTSYAMHWVRQAPGQRLEWMGWINAGNGNTKYSQKFQDRVTITRDTSASTAYMELSSLRSEDTAIYYCARDKVDDYGDYWFPTLWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 128 aa, Artificial sequence
  Synthetic peptide
EVQLVESGAEVKKPGASVKVSCKASGYTFTSYAMHWVRQAPGQRLEWMGWINAGNGNTKYSQKFQDRVTITRDTSASTAYMELSSLRSEDTAIYYCARDKVDDYGDYWFPTLWYFDYWGQGTLVTVSS

SEQ ID NO: 44 - protein, 8 aa, Artificial sequence
  Synthetic peptide
GYTFTSYA

SEQ ID NO: 45 - protein, 8 aa, Artificial sequence
  Synthetic peptide
INAGNGNT

SEQ ID NO: 46 - protein, 17 aa, Artificial sequence
  Synthetic peptide
ARDKVDDYGDYWFPTLW

SEQ ID NO: 47 - protein, 236 aa, Homo sapiens
MYRMQLLSCIALSLALVTNSQSALTQPASVSGSPGQSITISCTGTSSDVGGFNYVSWFQQHPGKAPKLMLYDVTSRPSGVSSRFSGSKSGNTASLTISGLQAEDEADYYCSSHTSRGTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 48 - protein, 110 aa, Artificial sequence
  Synthetic peptide
QSALTQPASVSGSPGQSITISCTGTSSDVGGFNYVSWFQQHPGKAPKLMLYDVTSRPSGVSSRFSGSKSGNTASLTISGLQAEDEADYYCSSHTSRGTWVFGGGTKLTVL

SEQ ID NO: 49 - protein, 9 aa, Artificial sequence
  Synthetic peptide
SSDVGGFNY

SEQ ID NO: 50 - protein, 2 aa, Artificial sequence
  Synthetic peptide
DV

SEQ ID NO: 51 - protein, 8 aa, Artificial sequence
  Synthetic peptide
SSHTSRGT

(19) United States
(12) Patent Application Publication
(10) Pub. No.: US 2023/0204567 A1
(43) Pub. Date: Jun. 29, 2023

(54) METHOD FOR DETERMINING THE POTENCY OF ANTIGENS

(71) Applicant: Takeda Vaccines, Inc., Cambridge, MA (US)

(72) Inventors: Jill LIVENGOOD, Cambridge, MA (US); Michael JOHNSON, Cambridge, MA (US); James GIFFORD, Cambridge, MA (US); Tawnya NEAD, Cambridge, MA (US); Mark LYONS, Cambridge, MA (US); Jackie MARKS, Cambridge, MA (US)

(73) Assignee: Takeda Vaccines, Inc., Cambridge, MA (US)

(21) Appl. No.: 17/999,131

(22) PCT Filed: Mar. 19, 2021
(86) PCT No.: PCT/US2021/023216

§ 371 Date: Nov. 17, 2022

Related U.S. Application Data
(60) Provisional application No. 63/027,553, filed on May 20, 2020.

(51) Int. Cl. G01N 33/542; G01N 33/533; G01N 33/569; G01N 33/68

(52) U.S. Cl. CPC G01N 33/542; G01N 33/533; G01N 33/56983; G01N 33/6878

(57) ABSTRACT

The present disclosure relates to a method for determining the potency of an antigen sample such as a vaccine antigen sample. The present disclosure is also related to a method for monitoring the potency of a vaccine antigen during the production process including purifying, inactivating and formulating the vaccine antigen and to a method for producing a virus vaccine. Further, the present disclosure relates to vaccines obtainable by the methods disclosed. In certain embodiments of the present invention the antigen sample is a zika virus antigen sample.

### CROSS REFERENCE TO RELATED APPLICATIONS

This International PCT Application claims priority to and the benefit of U.S. Provisional Application No. 63/027,553 filed on 20 May 2020, the contents of which is herein incorporated by reference in its entirety.

### SEQUENCE LISTING

This application incorporates by reference in its entirety the Sequence Listing entitled “T08498WO_PCTSequenceListing.txt” created on Mar. 16, 2021 at 4:18 pm that is 52 KB and filed electronically herewith.

### FIELD OF THE INVENTION

The present invention relates to a method, i.e. an immunoassay, for determining the potency of antigens as present in vaccines, including virus antigens. Further, the present invention is related to such a method for the application during vaccine production processes.

### BACKGROUND OF THE INVENTION

Viruses constitute a continually threat to human health. Fast adaption to changing environments and hosts enabled by high mutation rates complicate diagnosis, as well as prophylactic and therapeutic treatment of a manifold of virus infections. Moreover, approved vaccines are still missing for the prevention of diseases caused by several viruses.

A Zika virus (ZIKV) is an arthropod-borne virus (arbovirus) in the genus Flavivirus (family Flaviviridae) which also includes the West Nile virus (WNV), dengue virus (DENV), tick-borne encephalitis virus (TBEV), and yellow fever virus (YFV). It is thought to be principally transmitted to humans by the Aedes genus, i.e. by the mosquito Aedes aegypti ZIKV is classified into African and Asian genotypes by phylogenetic analysis.

Flaviviruses are enveloped, with icosahedral and spherical geometries. The diameter is around 50 nm. Genomes (10-11 kb bases) consists of linear positive-sense RNA and are non-segmented. The RNA is complexed with multiple copies of the capsid protein (C), surrounded by an icosahedral shell consisting of 180 copies each of the envelope glycoprotein (E protein; ˜500 amino acids), and the membrane protein (M protein; ˜75 amino acids) or precursor membrane protein (prM protein; ˜165 amino acids), all anchored in a lipid membrane. The genome also codes for seven non-structural proteins (NS1, NS2A, NS2B, NS3, NS4A, NS4B and NS5; WO2018010789).

As E protein is the main surface protein that participates in host cell receptor attachment and virus lipid bilayer fusion it is the major target of host neutralizing antibodies (Abs) against viral infection. The E protein is composed of an amino terminal ectodomain, two amphipathic α-helices and two carboxy terminal membrane-spanning α-helices. The surface-exposed ectodomain consists of three structurally distinct domains rich in β-sheets: a β-barrel domain I (EDI), a finger-like domain II (EDII), and a C-terminal domain III (EDIII).

As several epitopes are conserved among flaviviruses, antibody (Ab) responses to flavivirus infections are cross-reactive, hampering the diagnosis of a specific flavivirus infection for example by the determination of a flavivirus specific immune response. Cross-reactivity between ZIKV and other flavivirus Abs has been reported, particularly with DENV serocomplex due to high homology (54-59%) of the E protein. This comes even more into play as in a majority of ZIKV endemic regions, there are also DENV, including DENV serotypes 1 (DENV1), 2 (DENV2), 3 (DENV3), and 4 (DENV4), and other flaviviruses present, increasing the risk of multiple infections and therefore production of cross-reactive Abs. Further, the development of similar clinical manifestations by different flavivirus infections is of particular concern.

The potential effect of ZIKV as a public health threat increased due to isolated outbreaks in South-east Asia during 2007 and 2013 (Duffy et al., N Engl J Med. 2009, 360, 2536-2543; Hancock et al., Emerg. Infect. Dis. 2014, 20(11):1960). The largest ZIKV outbreak occurred in recent years when the spread reached to Brazil and throughout The Americas (Metsky et al., Nature 2017, 546(7658):411-415). ZIKV is associated with neurological sequelae and a broad spectrum of clinical manifestations and neonatal abnormalities known as the Congenital ZIKV Syndrome (CZS; Costello et al., Bull World Health Organ. 2016, 94(69):406-406A; Cao-Lormeau et al., Lancet 2016, Apr. 9; 387(10027): 1531-9).

Currently applied vaccine delivery platforms include live attenuated and inactivated whole-virus vaccines, viral-vectored vaccines utilizing for instance adeno-associated virus, DNA and mRNA vaccines, virus like particles (VLPs), and peptide and protein subunit vaccines (Maslow, Trop. Med. Infect. Dis. 2019, 4, 104). Although multiple vaccine candidates are currently evaluated in clinical trials, no treatment is approved yet for ZIKV (Poland et al., Mayo Clinic Proceedings 2019, 94, 2572-2586). A promising candidate is Takeda's purified inactivated Zika vaccine (PIZV) derived from PRVABC59, an American outbreak strain of Asian genotype. Purified inactivated vaccines have been successfully developed and safely utilized for the prevention of diseases caused by other flaviviruses, including JEV and TBEV (Ishikawa et al., Vaccine 2014, 32, 1326-1337).

However, as antigenicity, immunogenicity, and potency of an antigen as present in vaccines dependent on the availability of certain epitopes on the antigen surface, methods for fast, robust, and reliable characterization of antigens are of urgent need. Moreover, such methods will be beneficial for the surveillance of vaccine manufacturing processes, as the corresponding antigens can be monitored during the pipeline of production.

### OBJECTS AND SUMMARY

It is an object of the present invention to provide a method for determining the potency of an antigen sample such as a vaccine antigen sample or a virus antigen sample.

It is a further object of the present invention to provide a method for determining the potency of an antigen sample, the assay providing good specificity.

It is a further object of the present invention to provide a method for determining the potency of a virus antigen sample such as an inactivated virus or live virus.

It is a further object of the present invention to provide a method for determining the potency of an antigen sample, the assay showing no cross-reactivity with other antigens, such as antigens of other viruses.

It is a further object of the present invention to provide a method for determining the potency of an antigen sample, the method providing good sensitivity thereby for instance enabling analyzing low sample amounts.

It is a further object of the present invention to provide a method for determining the potency of an antigen sample, the method providing a simple operation and rapid detection (e.g. no washing are steps required during the procedure).

It is a further object of the present invention to provide a method for determining the potency of an antigen sample, the method providing a homogenous assay format, a robust performance and a low background signal.

It is a further object of the present invention to provide a method for determining the potency of an antigen sample, the method providing low detection costs due to small sample volumes.

It is a further object of the present invention to provide a method for determining the potency of an antigen sample, the method providing a low detection limit and a wide dynamic range.

It is a further object of the present invention to provide a method for determining the potency of an antigen sample, the method providing a low false-positive and a low false-negative rate.

It is a further object of the present invention to provide a method for determining the potency of an antigen sample, the method providing the potential for high-throughput application, for instance, during manufacturing processes of vaccines.

It is a further object of the present invention to provide a method for producing a virus vaccine comprising the application of the method for determining the potency of an antigen sample for determining the potency of the vaccine antigen and thereby monitoring the steps of the production process of the vaccine.

It is a further object of the present invention to provide a vaccine obtainable by the method for determining the potency of an antigen sample as present in vaccines.

It is a further object of the present invention to provide a kit, comprising an acceptor and a donor antibody, as well as an acceptor and a donor microsphere suitable for the application in the method for determining the potency of a zika virus antigen.

Therefore the invention is directed to a method for detecting a signal indicative for the potency of an antigen sample such as a vaccine antigen sample, wherein the antigen in the antigen sample provides at least two epitopes and the method comprises the steps of:
Step 1: providing a kit comprising an acceptor kit and a donor kit, the acceptor kit comprising an amount of an acceptor microsphere and an amount of an acceptor antibody and the donor kit comprising an amount of a donor microsphere and an amount of a donor antibody, wherein
   the acceptor microsphere is capable to accept energy which is transferred in a proximity reaction to produce a signal and is capable of binding or is bound to the constant region of the acceptor antibody and is not capable of binding to the donor antibody,
   the acceptor antibody has a variable region which is capable of binding to one of the at least two epitopes of the antigen and a constant region which is capable of binding or is bound to said acceptor microsphere, wherein the acceptor antibody is not capable of binding to the donor microsphere,
   the donor microsphere is capable to donate energy which is transferred in a proximity reaction to produce a signal by the acceptor microsphere and is capable of binding or is bound to the constant region of the donor antibody and is not capable of binding to the acceptor antibody, and
   the donor antibody has a variable region which is capable of binding to the other of the at least two epitopes of the antigen and a constant region which is capable of binding to said donor microsphere, wherein the donor antibody is not capable of binding to the acceptor microsphere,
Step 2: contacting the amount of said donor microsphere, the amount of said acceptor microsphere, the amount of said donor antibody and the amount of said acceptor antibody of step 1 with the sample to allow forming a complex of the antigen in the sample with the donor antibody bound to the donor microsphere and the acceptor antibody bound to the acceptor microsphere and the acceptor antibody bound to one of the at least two epitopes of the antigen and the donor antibody bound to the other of the at least two epitopes of the antigen,
Step 3: conducting a proximity reaction to produce a signal indicative for the potency of the antigen sample, and
Step 4: detecting the signal indicative for the potency of the antigen sample.

The present invention is further directed to a such a method for determining the amount of the antigen in the antigen sample indicative for the potency of the antigen sample by detecting the signal in accordance with the method as described above and further comprising the step of:
Step 5: determining the amount of the antigen in the antigen sample indicative for the potency of the antigen sample based on the detected signal.

The present invention is further directed to such a method for determining the potency of the antigen sample such as a vaccine antigen sample by detecting the amount of the antigen in accordance with the method as described above and further comprising the step of:
Step 6: determining the potency of the antigen sample based on the amount of the antigen in the sample determined in step 5.

The present invention is further directed to a method of producing a virus vaccine comprising the steps of:
Step A: preparing various batches of vaccine antigen,
Step B: determining the potency of the vaccine antigen of the various vaccine antigen batches produced in step A in accordance with the method as described above and selecting the vaccine antigen batches in conformity with a predetermined potency requirement,
Step C: preparing vaccine batches by formulating the vaccine antigen batches selected in step B into various batches of virus vaccine,
Step D: determining the potency of the vaccine antigen in the vaccine batches of the various batches produced in step C in accordance with the method as described above and selecting the vaccine batches in conformity with the predetermined potency requirement.

The present invention is further directed to a vaccine obtainable by the method as described above.

The present invention is further directed to a kit comprising an acceptor kit and a donor kit, the acceptor kit comprising an amount of an acceptor microsphere and an amount of an acceptor antibody and the donor kit comprising an amount of a donor microsphere and an amount of a donor antibody, wherein
the acceptor microsphere is capable to accept energy which is transferred in a proximity reaction to produce a signal and is capable of binding or is bound to the constant region of the acceptor antibody and is not capable of binding to the donor antibody,
the acceptor antibody has a variable region which is capable of binding to one of the at least two epitopes of a zika virus antigen and a constant region which is capable of binding or is bound to said acceptor microsphere, wherein the acceptor antibody is not capable of binding to the donor microsphere,
the donor microsphere is capable to donate energy which is transferred in a proximity reaction to produce a signal by the acceptor bead and is capable of binding or is bound to the constant region of the donor antibody and is not capable of binding to the acceptor antibody, and
the donor antibody has a variable region which is capable of binding to the other of the at least two epitopes of the zika virus antigen and a constant region which is capable of binding to said donor microsphere, wherein the donor antibody is not capable of binding to the acceptor microsphere.

The invention is further directed to a method for determining the potency of the antigen sample as described above, wherein the antigen is a zika antigen and the kit is defined as described above.

The invention is further directed to a zika antigen, obtainable by the method as described above.

### ABBREVIATIONS AND DEFINITIONS

### Abbreviations

“ZIKV” refers to zika or zika virus. “DENV” refers to dengue or dengue virus. “DENV1” refers to dengue virus serotype 1. “DENV2” refers to dengue virus serotype 2. “DENV3” refers to dengue virus serotype 3. “DENV4” refers to dengue virus serotype 4. “VLP” refers to virus like particle. “E protein” refers to envelope glycoprotein. “EDI”, “EDII”, “EDIII” refer to domain I, II, and III of the E protein. “M protein” refers to membrane protein. “prM” refers to precursor membrane protein. “RFU” refers to relative fluorescent units. “Ab” and “Abs” stand for antibody and antibodies. “Ig” stands for immunoglobulin. “mAb” stands for monoclonal antibody. “Anti-ZIKV Ab” refers to an Ab that binds to a ZIKV antigen. “CDR” stands for complementary determining region. “RVP” refers to reporter virus particle. “TCID₅₀” refers to 50% tissue culture infectious dose. “ZAPA” refers to zika antigen potency assay. “PIZV” refers to purified inactivated zika vaccine. “PRNT” refers to plaque reduction neutralization test. “MNT” refers to microneutralization test. “FFA” refers to focus forming assay. “PFU” refers to plaque forming units. “FFU” refers to focus forming units. “AU” refers to antigen units.

### Definitions

As used in the present disclosure and claims, the singular forms “a,” “an,” and “the” are to be construed to cover both the singular and the plural forms unless the context clearly dictates otherwise.

The term “and/or” as used in a phrase such as “A and/or B” herein is intended to include “A”, “B”, and “A and B”.

Open terms such as “include”, “including”, “contain”, “containing” and the like mean “comprising”. These open-ended transitional phrases are used to introduce an open ended list of elements, method steps, or the like that does not exclude additional, unrecited elements or method steps.

As used herein, the terms “antibody (Ab)” or “antibodies (Abs)” refer to an immunoglobulin (Ig) molecule, generally comprised of four polypeptide chains, two heavy (H) chains and two light (L) chains interconnected by disulfide bonds (full length Ab) and includes any naturally occurring, enzymatically obtainable, synthetic, or genetically engineered polypeptide or glycoprotein that specifically binds an antigen to form a Ab/antigen complex. Abs can be obtained using standard recombinant DNA techniques. In a full length Ab, each heavy chain is comprised of a heavy chain variable region (VH) and a heavy chain constant region (CH). The heavy chain constant region is comprised of three domains, CH1, CH2 and CH3. Each light chain is comprised of a light chain variable region (VL) and a light chain constant region (CL). The light chain constant region is comprised of one domain. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In certain embodiments of the present invention, the FRs of the Ab may be identical to the human germline sequences, or may be naturally or artificially modified. The terms Ab or Abs may also refer to any functional fragment, mutant, variant, or derivative thereof. Such functional fragment, mutant, variant, or derivative antibody formats are known in the art. Ab fragments such as Fab or F(ab′)2 fragments, can be prepared from full length Abs using conventional techniques such as papain or pepsin digestion, respectively, of full length Abs. Functional fragments are in particular (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1), which comprises a single variable domain; and (vi) an isolated CDR. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). In certain embodiments, scFv molecules may be incorporated into a fusion protein. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such functional fragments are known in the art (Kontermann and Dubel eds., Antibody Engineering (2001) Springer-Verlag. New York. 790 pp. (ISBN 3-540-41354-5)). The Ab may be described by the term “anti-antigen Ab” to express to which antigen the Ab is able to bind. For instance, an “anti-ZIKV Ab” refers to an Ab that binds to a ZIKV antigen. Ab or Abs may be mono-specific, bi-specific, or multi-specific. Multi-specific Abs may specifically bind different epitopes of one antigen or may specifically bind two or more unrelated antigens. See, e.g., Tut et al., 1991, J. Immunol. 147:60-69; Kufer et al., 2004, Trends Biotechnol. 22:238-244. Abs including any of the multi-specific antigen-binding molecules of the present invention, or variants thereof, may be constructed using standard molecular biological techniques (e.g., recombinant DNA and protein expression technology), as will be known to a person of ordinary skill in the art, for instance intracellular expression systems. Abs may be multivalent Abs comprising two or more antigen binding sites. Substitution of one or more CDR residues or omission of one or more CDRs is also possible. Abs have been described in the scientific literature where one or two CDRs can be dispensed with barely an effect for binding. Analysis of the contact regions between Abs and their antigens, based on published crystal structures, revealed that only about one fifth to one third of CDR residues actually contact the antigen. Moreover, many Abs have one or two CDRs were no amino acids are in contact with an antigen (Padlan et al. FASEB J. 1995, 9: 133-139, Vajdos et al., J Mol Biol 2002, 320:415-428). CDR residues not contacting antigen can be identified based on previous studies (for example residues H60-H65 in CDR2 of the heavy chain are often not required), from regions of Kabat CDRs lying outside Chothia CDRs, by molecular modeling and/or empirically. If a CDR or residue(s) thereof is omitted, it is usually substituted with an amino acid occupying the corresponding position in another human Ab sequence or a consensus of such sequences. Positions for substitution within CDRs and amino acids to substitute can also be selected empirically. Empirical substitutions can be conservative or non-conservative substitutions. The terms Ab or Abs may refer to Ab or Abs that originate from certain origin species that for example include rabbit, mouse, human, monkey, or rat (rabbit Ab, mouse Ab, human Ab, monkey Ab, or rat Ab). For instance, rabbit origin may be intended to include Abs having variable and constant regions derived from rabbit germline immunoglobulin sequences. Abs may comprise one or more amino acid substitution, insertion, and/or deletion as compared to corresponding germline sequences. The Abs may also include amino acid residues not encoded by the origin species germline immunoglobulin sequences (e.g. mutations introduced by random or site-specific mutagenesis in vitro or in vivo), for example in the CDRs. As used herein, an Ab or Abs originating from a certain origin species (e.g. rabbit) may also refer to an Ab or Abs in which CDR or other sequences derived from the germline of another mammalian species (e.g. mouse) have been grafted onto the origin species (e.g. rabbit) framework region (FR) sequences. Abs may be chimeric Abs. Chimeric Abs may encompass sequences derived from the germline of different species and may also include further amino acid substitutions or insertions. Abs may be humanized Abs that are human immunoglobulins that contain minimal non-human (e.g., murine) sequences. Typically, in humanized antibodies residues from the human CDR are replaced by residues from the CDR of a non-human species (e.g., mouse, rat, rabbit, and hamster, etc.; Jones et al., Nature 1986; 321:522-525; Riechmann et al., Nature 1988, 332:323-327; Verhoeyen et al., Science 1988, 239:1534-153). Non-limiting examples of methods used to generate humanized antibodies are described in U.S. Pat. No. 5,225,539; Roguska et al., Proc. Natl. Acad. Sci. 1994, USA 91:969-973; and Roguska et al., Protein Eng. 1996; 9:895-904. Abs can be of any class (e.g., IgG, IgE, IgM, IgD, IgA and IgY) and subclass (isotype) (e.g., IgG1, IgG2, IgG3, IgG4, IgA1 and IgA2). In some embodiments, the immunoglobulin is an IgG1 isotype. In some embodiments, the immunoglobulin is an IgG2 isotype. The different classes of immunoglobulins have different and well-known subunit structures and three-dimensional configurations. Abs may comprise sequences from more than one class or subclass. Abs may be free of other Abs having different antigenic specificities (e.g. an Ab that binds ZIKV is substantially free of Abs that bind antigens other than ZIKV). The Ab may be free of other cellular material and/or chemicals. The terms Ab or Abs may refer to a neutralizing or non-neutralizing Ab. The terms Ab or Abs may refer to a monoclonal Ab. The terms Ab or Abs may refer to a recombinant Ab. The term Ab or Abs may refer to a donor Ab. The term Ab or Abs may refer to an acceptor Ab.

As used herein, the term “constant region” of an Ab refers to the heavy chain constant region (CH) and/or the light chain constant region (CL).

As used herein, the term “variable region” of an Ab refers to the heavy chain variable region (VH) and/or the light chain variable region (VL).

As used herein, the term “binds to”, “is binding to”, or “capable of binding to” refers within the context of an Ab that binds to or is binding to or is capable of binding to, to an Ab that is able to bind a certain molecule e.g. a microsphere or an antigen. Ability of binding to a certain antigen can be investigated by methods well known in the art including enzyme linked immunosorbent assay (ELISA), or bio-layer interferometry (BLI). Thereby, the Ab provides a signal above the background or noise of the method when tested for binding to the antigen. In certain embodiments, the Ab provides a signal when tested for binding to the antigen, which is at least 10%, at least 25%, at least 35%, at least 50%, at least 60%, at least 75%, at least 85%, at least 90%, at least 95%, or at least 100% higher than the signal the Ab provides when tested for binding to comparable antigens. In a specific embodiment the antigen is a ZIKV antigen (i.e. a ZIKV vaccine) and the comparable antigens are DENV antigens. The Ab can be able to bind to said molecule with the Ab constant region or variable region. In the case that the molecule is an antigen the Ab is able to bind to the antigen with the antibody variable region. In the case that the molecule is a microsphere, the Ab is able to bind to the microsphere with the Ab constant region.

As used herein, the term “is bound to” refers within the context of an Ab that is bound to, to an Ab that is bound to a molecule e.g. a microsphere, or an antigen. The Ab can be bound to said molecule with the antibody constant or variable region. In the case that the molecule is an antigen the Ab is bound to the antigen with the antibody variable region. In the case that the molecule is a microsphere, the Ab is bound to the microsphere with the antibody constant region. Consequently, as used herein, the term “is bound to” refers within the context of a microsphere that is bound to, to a microsphere that is bound to the constant region of an Ab. The Ab can be covalently bound to the microsphere and vice versa (“is covalently bound to”).

As used herein, the term “allow forming a complex” refers within the context of a donor Ab, a donor microsphere, an acceptor Ab, an acceptor microsphere, and a sample to a situation, wherein an amount of said donor microsphere, an amount of said acceptor microsphere, an amount of said donor antibody and an amount of said acceptor antibody is contacted with a sample for a sufficient time to enable formation of a complex of the antigen in the sample with the donor antibody bound to the donor microsphere and the acceptor antibody bound to the acceptor microsphere and the acceptor antibody bound to one of the at least two epitopes of the antigen and the donor antibody bound to the other of the at least two epitopes of the antigen. If the donor and acceptor Ab are not binding and/or bound to the antigen in the sample, no complex will be formed.

As used herein, the term “complementary determining region (CDR)” refers to the CDR within the Ab variable sequences. There are three CDRs in each of the variable regions of the heavy chain (VH) and the light chain (VL), which are designated CDR1, CDR2 and CDR3 (or specifically VH-CDR1, VH-CDR2, VH-CDR3, VL-CDR1, VL-CDR2, and VL-CDR3), for each of the variable regions. The term CDR may refer to a group of three CDRs that occur in a single variable region capable of binding the antigen. The exact boundaries of these CDRs can be defined differently according to different systems. The system described by Kabat (Kabat et al., Sequences of Proteins of Immunological Interest (National Institutes of Health, Bethesda, Md. (1987) and (1991)) refers to an unambiguous residue system applicable to any variable region of an antibody, but also provides precise residue boundaries defining the three CDRs. These CDRs may be referred to as Kabat CDRs. For the VH region, the hypervariable region ranges from amino acid positions 31 to 35 for VH-CDR1, amino acid positions 50 to 65 for VH-CDR2, and amino acid positions 95 to 102 for VH-CDR3. For the VL region, the hypervariable region ranges from amino acid positions 24 to 34 for VL-CDR1, amino acid positions 50 to 56 for VL-CDR2, and amino acid positions 89 to 97 for VL-CDR3. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “framework”, “framework region (FR)” or “framework sequence” refers to the remaining sequences of a variable region minus the CDRs. Because the exact definition of a CDR sequence can be determined by different systems, the meaning of a framework sequence is subject to correspondingly different interpretations. The six CDRs (VL-CDR1, VL-CDR2, and VL-CDR3 and VH-CDR1, VH-CDR2, and VH-CDR3) also divide the framework regions on the light chain (L) and the heavy chain (H) into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

A “recombinant Ab”, as used herein, refers to an Ab which is created, expressed, isolated or obtained by technologies or methods known in the art such as recombinant DNA technology which include, e.g. DNA splicing and transgenic expression. The term may refer to Abs expressed in a non-human mammal (including transgenic non-human mammals e.g. transgenic mice), or a cell (e.g. CHO cells) expression system or isolated from a recombinant combinatorial human antibody library.

A “neutralizing Ab”, as used herein, is intended to refer to an Ab which provides a titer above the lower limit of detection and/or the background in a microneutralization test (MNT), plaque reduction neutralization test (PRNT), a focus forming assay (FFA) and/or reporter virus particle (RVP) test. A neutralizing Ab may be used alone or in combination as prophylactic or therapeutic agent with other anti-viral agents upon appropriate formulation, or in association with active vaccination, or as a diagnostic tool. The term neutralizing Ab may refer to an Ab which prevents, inhibits, reduces, impedes, or interferes with the ability of a pathogen e.g. a ZIKV to initiate and/or perpetuate an infection in a host. The epitope to which a neutralizing Ab binds to may be referred to as a “neutralizing epitope”.

As used herein, the term “antibody titer” refers to a certain amount of Ab within a sample. The sample may be a blood plasma, urine, blood, or serum sample. An antibody titer can be expressed as the inverse of the highest dilution (in a serial dilution row) that still gives a positive test result. Consequently the term “neutralizing antibody titer” refers to a certain amount of neutralizing Abs within a sample. An Ab titer or neutralizing Ab titer can be determined by various method well known in the art including enzyme linked immunosorbent assay (ELISA), microsphere immunoassays, RVP assay, MNT, FFA, or PRNT.

As used herein, the term “immunoassay” refers to an assay that detects, determines, identifies, characterizes, quantifies, or otherwise measures the presence and/or concentration of a molecule through the use of an Ab or antigen. The molecule detected by the immunoassay can be present in biological samples (e.g. serum or blood plasma). The molecule detected by the immunoassay may be itself an Ab or antigen.

As used herein, the term “microsphere immunoassay” refers to an assay that detects, determines, identifies, characterizes, quantifies, or otherwise measures the presence and/or concentration of Abs with the use of microspheres coupled to an antigen to which the Abs are able to bind. The Abs detected by the microsphere immunoassay can be present in biological samples (e.g. serum or blood plasma).

As used herein, the term “reporter virus particle (RVP)” refers to particles that retain the antigenic determinants of wild-type virions and include capsid (C), envelope (E), pre-membrane (prM) and membrane (M) proteins. Upon infection of cells with RVPs a reporter gene e.g. Renilla luciferase or firefly luciferase is expressed. RVPs enable tracking of a virus infection over time and quantifying events such as virus cellular entry and replication.

As used herein, the term “reporter virus particle assay (RVP assay)” or “reporter virus particle test (RVP test)” refers to an assay for determining neutralizing Ab titers in a sample. Thereby, cells as for instance Vero cells, are incubated with the sample, followed by the addition of RVPs. The half maximal effective concentration (EC₅₀) titer of neutralizing Abs is determined by addition of a suitable substrate that is converted by the reporter gene expressed upon RVP infection to a create detectable signal. For instance, upon conversion of the substrate coelenetrazine, luciferase produces a luminescence signal that can be detected. Reduction of the luminescence signal compared to a control lacking the sample, is an indicator for the presence and/or the amount of neutralizing Abs within the sample.

As used herein, the term “cytophatic effects (CPE)” refers to visible changes induced upon virus infection of monolayer culture cells as for instance Vero cells. CPE include rounding and detaching of cells from the culture plate. CPE can be observed with a light microscope or by a spectrometric readout. The spectrometric readout is based on the fact that cell death upon virus infection causes the cell media pH to change. This pH change can be visualized by the application of indicators within the cell media (e.g. phenol red) and detected by measuring the absorbance at about 560 nm and about 420 nm and comparing these two values.

As used herein, the term “microneutralization test (MNT)” refers to a method for determining neutralizing Ab titers in a sample. By mixing the virus with a serial dilution of the sample the reduction of CPE can be observed and thereby the amount of neutralizing Abs within the sample can be determined.

As used herein, the term “endpoint dilution assay” refers to a method for measuring infectious virus titers within a sample. The method relies on the occurrence of CPE upon incubation of monolayer cells as for instance Vero cells with a serial dilution of a sample. After incubation, CPE is determined for each sample dilution and results are used to mathematically calculate a fifty-percent tissue culture infectious dose (TCID₅₀) result. The TCID₅₀ refers within that context to the amount of virus required to produce a cytopathic effect in 50% of inoculated tissue culture cells. Methods for calculation of the TCID₅₀ commonly used include the method of Reed and Muench and the method of Spearman and Karber.

As used herein, the term “plaque reduction neutralization test (PRNT)” refers to a test for determining neutralizing Ab titers for a virus. Therefore, the sample (e.g. serum) is diluted and mixed with a certain amount of virus. Afterwards, the mixture is applied onto confluent monolayer cells (e.g. Vero cells). The surface of the cell layer is subsequently covered with a layer of semisolid overlay medium as for instance agar to prevent the virus from spreading indiscriminately. The concentration of plaque forming units (PFU) can be estimated by the number of plaques (regions of lysed cells) formed after a few days. Depending on the virus, the plaque forming units are measured by microscopic observation and/or specific dyes that react with infected cells or solely stain living cells (e.g. crystal violet). The concentration of sample to reduce the number of plaques by 50% compared to the control, wherein the cells are infected with virus only (lacking addition of sample) is denoted as the “PRNT50” value.

As used herein, the term “focus forming assay (FFA)” refers to a variation of the PRNT assay, wherein the regions of infected cells (foci) are detected by fluorescent antibodies specific for a viral antigen to detect infected host cells and infectious virus particle. The FFA is particularly useful for quantifying classes of viruses that do not lyse the cell membranes, as these viruses would not be amenable to the PRNT. The result of the FFA is reported as focus forming units (FFU).

As used herein, the term “plaque forming units (PFU)” refers to the number of virus particles capable of forming plaques (regions of lysed cells) per unit volume.

As used herein, the term “focus forming units (FFU)” refers to the number of virus particles capable of forming foci (regions of infected cells) per unit volume.

As used herein, the term “enzyme linked immunosorbent assay (ELISA)” refers to an immunoassay for the measurement of Abs and antigens depending on the specific set-up. A key feature of all ELISA set-ups is the application of a plate on which Abs or antigens are immobilized. For instance, in order to determine Abs within a sample, a corresponding antigen to which the Abs bind to is immobilized on the plate. In another set-up, Abs are immobilized on the plate to detect antigens within a sample. The signal of an ELISA is generated by an enzymatic reaction producing a signal that can be for instance detected by spectrophotometric methods. A common example of an enzyme applied is horseradish peroxidase. Common ELISA set-ups include direct ELISA, sandwich ELISA, competitive ELISA, and reverse ELISA.

As used herein, the term “monoclonal Ab” (“mAb”) refers to an Ab obtained from a population of substantially homogenous Abs that bind to the same antigenic determinants (epitopes). “Substantially homogeneous” means that the individual Abs are identical except for possibly naturally-occurring mutations that may be present in minor amounts. This is in contrast to polyclonal antibodies that typically include different antibodies directed against various, different antigenic determinants (epitopes). A monoclonal Ab may be generated by hybridoma technology according to methods known in the art (Köhler and Milstein, Nature 1975, 256:495-497), phage selection, recombinant expression, and transgenic animals.

As used herein, the term “does not cross-react” refers to an Ab that does not bind to a certain antigen e.g. a flavivirus or a DENV. “Does not bind” within that context means that the Ab shows a binding signal when tested for binding to a certain antigen e.g. a flavivirus or DENV that is 30% or less, 20% or less, more preferable 10% or less, even more preferable 5% or less of the binding signal when the Ab is tested for binding to another antigen e.g. a ZIKV. In certain embodiments, “does not bind” within that context means that the Ab does not show a binding signal above the background signal and/or lower limit of detection when tested for binding to the antigen e.g. a flavivirus or DENV. For instance, suitable methods for detecting a binding signal include enzyme linked immunosorbent assay (ELISA) or microsphere immunoassays using the corresponding antigen.

As used herein, the term “detection system” refers to any system which is suitable for determining the signal produced by a proximity reaction. The term detection system may additionally refer to a system which is suitable for exciting the molecule within the donor microsphere thereby initiating the proximity reaction and determining the signal produced by this proximity reaction

A “recombinant protein”, as used herein, refers to a protein which is created, expressed, isolated or obtained by technologies or methods known in the art such as recombinant DNA technology which include, e.g. polymerase chain reaction (PCR), DNA splicing and transgenic expression. The term may refer to proteins expressed in a non-human mammal (including transgenic non-human mammals e.g. transgenic mice), or a cell (e.g. human embryonic kidney cells (HEK293), Chinese hamster ovary (CHO) cells, or bacterial cells like Escherichia coli) expression system. The recombinant protein may be purified by protein purification methods known in the art such as immobilized metal affinity chromatography (IMAC; e.g. His-purification) and size-exclusion chromatography. The protein may be characterized by methods known in the art such as e. g. Bradford or bicinchoninic acid (BCA) assays for determination of protein concentration, or biolayer interferometry (BLI) for determination of binding properties of the protein.

As used herein, the terms “microsphere” or “microspheres” refer to a small particles that can be bound to molecules like antibodies (Abs) for use in the methods of the present invention. The terms microsphere, particle, microparticle, bead, or microbead can be used interchangeably and bear equivalent meanings. The microsphere is capable to either donate or accept energy which is transferred in a proximity reaction.

As used herein, the term “antigen” refers to any substance which can be bound by an Ab. Antigens may induce an immune response within a subject. An antigen may have one or more epitopes. Thus, different Abs may bind to different areas on the antigen. An antigen may be a protein, polypeptide, carbohydrate, polynucleotide, lipid, or combinations thereof. The antigen may be a virus antigen, for instance a ZIKV antigen, a DENV antigen, a poliovirus antigen, or a norovirus antigen. The virus antigen may also be a live virus, an inactivated virus, a live attenuated virus or a virus like particle. The antigen may be a vaccine antigen (an antigen which is present in vaccines), which can be itself a virus antigen. A vaccine antigen can be inactivated (e.g. by formaldehyde treatment) and formulated to a vaccine.

As used herein the term “ZIKV antigen”, refers to any antigen which is a, is part of or is derived from a ZIKV. Examples include but are not limited to native ZIKV, inactivated ZIKV (e.g. heat-inactivated, formaldehyde-inactivated), attenuated ZIKV, ZIKV virus-like particles (VLPs), ZIKV structural or non-structural proteins, ZIKV NS1 protein, ZIKV E protein, ZIKV E protein domain III (EDIII), a ZIKV immunogenic composition (e.g. a ZIKV vaccine), any precursor of a ZIKV immunogenic composition or any combination thereof. The ZIKV vaccine may be a purified inactivated ZIKV vaccine (PIZV). The PIZV may be adsorbed on alum.

As used herein, the terms “subject” or “subjects” can include any individual. A subject may be, but is not limited to, a mouse, a primate, a non-human primate (NHP), a human, a rabbit, a cat, a rat, a horse, a sheep. In certain embodiment the subject can be a pregnant mammal, and in particular embodiments a pregnant human female. In some embodiments the subject is a patient, for whom prophylaxis or therapy is desired.

As used herein, the term “non-human subject” can include any individual that is not a human. A non-human subject may be, but is not limited to, a mouse, a primate, a non-human primate (NHP), a rabbit, a cat, a rat, a horse, a sheep.

As used herein, the term “sample” refers to a sample that can be of any origin. The sample may be derived from a subject. Samples may include but are not limited to body fluids (like serum, blood, urine, cerebrospinal fluid, lymph fluid), immunogenic compositions (like vaccines, or any precursors thereof), and cell culture components (like cell culture supernatants, cell lysates). A sample may be an antigen sample (e.g. a vaccine antigen sample, a virus antigen sample). In the case a sample is a body fluid, it is required that the sample is a sample outside the human or animal body. Samples may contain any kind of analyte such as vaccine antigen or virus antigen (vaccine antigen sample, virus antigen sample). The term sample may also refer to samples from different stages of a manufacturing process of a vaccine. The term sample may also refer to different vaccine batches. The term sample may also refer to different vaccine batches containing antigens as ZIKV antigens in different quality. The term may also refer to different vaccine batches containing antigens as ZIKV antigens and additional components such as alum. The said sample can be pre-treated prior to use, such as preparing plasma from blood, diluting fluids, or the like. Methods for pre-treating can involve purification, filtration, distillation, concentration, inactivation of interfering compounds, and the addition of reagents. In some embodiment the sample is heat-inactivated and/or inactivated with formaldehyde.

As used herein, the term “antigen sample” refers to any sample which contains a certain amount of antigen. Consequently, the term “virus antigen sample” refers to any sample which contains a virus antigen. Consequently, the term “vaccine antigen sample” refers to any sample which contains a vaccine antigen, such as a virus antigen.

As used herein, the term “batch” refers to a certain sample (e.g. an antigen sample) or a certain batch of vaccine. Different batches may for instance differ in their quality. For instance, one vaccine batch may show a higher potency than another vaccine batch.

As used herein, the term “standardized sample” or “standardized antigen sample” refers to a characterized sample. Standardized samples can be applied to establish a standard curve for determination of the potency of test samples, by determining the potency of standardized samples in any subject as well as determining the amount of antigen in the standardized samples in the same way as the amount of antigen in the test sample is determined and plotting the amount of antigen in the standardized samples against the determined corresponding potency. The potency of the standardized samples can be expressed as mean neutralizing antibody titers. Standardized samples can be provided by a forced degradation study or by application of different doses of an antigen (e.g. serial dilutions of a stock standardized sample).

Within the context of this invention the term “forced degradation study” refers to any study which provides and/or uses samples containing an antigen, wherein the antigen is degraded to a different degree in a controlled way. For instance, degradation can be carried out by pH changes (e.g. acidification of samples) or heat-degradation (e.g. incubation of samples at about 56° C. for about 30 to about 60 min). For instance, a sample can be heat-degraded and certain amounts of this heat-degraded sample can be mixed with a non-degraded sample to result in 25%, 50%, 75%, and 100% heat-degraded sample.

As used herein, the term “dose” of antigen refers to a certain amount of an antigen e.g. expressed as an absolute amount (such as mg, μg, and ng) or as a concentration (such as mg/mL, μg/mL, and ng/μL).

As used herein, the term “antigenicity” refers to the capacity of an antigen to bind to Abs and therefore to the availability of certain epitopes. Antigenicity is measured by in vitro conformational methods as for instance the methods disclosed in the present invention.

As used herein, the terms “potency” and “immunogenicity” refer to the ability of an antigen (e.g. as present in antigen samples such as vaccine antigen samples) and/or a vaccine to induce an immune response in a subject (e.g. a human or a model animal as a mouse). The immune response can be humoral and/or cell-mediated. In comparison with antigenicity, potency and immunogenicity are measured by in vivo studies administering the antigen or the vaccine to a subject and monitoring the induced immune response.

As used herein, the terms “virus like particle (VLP)” or “virus like particles (VLPs)” refer to molecules that closely resemble viruses, but are non-infectious because they do not contain viral genetic material. VLPs can be prepared recombinant through the expression of viral structural proteins, which can then self-assemble into the VLPs. Consequently, ZIKV VLP refers to a VLP comprising ZIKV structural proteins. VLPs can be used as vaccines to induce an immune response in a subject.

As used herein, the term “E protein” refers to the envelope glycoprotein (E). Consequently “ZIKV E protein” refers to ZIKV envelope glycoprotein (E). The E protein may be a recombinant protein. The amino acid sequence of ZIKV E protein is part of the viral polyprotein encoded by a ZIKV strain. In particular, the amino acid sequence of ZIKV E protein (SEQ ID NO: 1) is part of the viral polyprotein (E protein corresponds to amino acids 291-794; GenBank accession No. AWH65849.1) encoded by the ZIKV strain PRVABC59 (GenBank accession No. MH158237.1).

As used herein, the term “EDIII” refers to ZIKV carboxyl (C)-terminal domain III of the E protein ectodomain. The amino acid sequence of EDIII protein is part of the E protein, which is part of the viral polyprotein encoded by a ZIKV strain. For instance, the amino acid sequence of EDIII is encoded within SEQ ID NO: 1 of ZIKV strain PRVABC59 (GenBank Accession No. MH158237.1).

As used herein, the term “epitope” or “antigenic determinant” refers to the part of an antigen that interacts with a specific antigen-binding site in the variable region of an Ab molecule known as a paratope. Conversely, the “epitope” can also interact with a specific cellular receptor or binding site on a host. A single antigen may have more than one epitope. Thus, different Abs may bind to different areas on an antigen and may have different biological effects. For example, the term “epitope” also refers to a site on an antigen to which B and/or T cells respond. Epitopes may be defined as structural or functional. Functional epitopes are generally a subset of the structural epitopes and have those residues that directly contribute to the affinity of the interaction. The epitope to which the antibodies bind may consist of a single contiguous sequence of 2 or more (e.g., 2, 3, 4, 5, 6, 7, 8, 9, 10, 11, 12, 13, 14, 15, 16, 17, 18, 19, 20 or more) amino acids located within an antigen i.e. a linear epitope for instance in a domain of a ZIKV E protein. Epitopes may also be conformational, that is, composed of a plurality of non-contiguous amino acids, i.e., non-linear amino acid sequence. A conformational epitope typically includes at least 3 amino acids, and more commonly, at least 5 amino acids, e.g., 7-10 amino acids in a unique spatial conformation. In certain embodiments, epitopes may include determinants that are chemically active surface groupings of molecules such as amino acids, sugar side chains, phosphoryl groups, or sulfonyl groups, and, in certain embodiments, may have specific charge characteristics. Epitopes formed from contiguous amino acids are typically retained on exposure to denaturing solvents, whereas epitopes formed by tertiary folding are typically lost on treatment with denaturing solvents. Various techniques known to persons of ordinary skill in the art can be used to determine whether an antibody interacts with one or more amino acids within a polypeptide or protein. Exemplary techniques include, for example, site-directed mutagenesis (e.g., alanine scanning mutational analysis). Other methods include routine cross-blocking assays (such as that described in Antibodies, Harlow and Lane, Cold Spring Harbor Press, Cold Spring Harbor, NY), peptide blot analysis (Reineke (2004) Methods Mol. Biol. 248: 443-63), peptide cleavage analysis crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues that correspond to the specific amino acids with which the antibody interacts. See, e.g., Ehring (1999) Analytical Biochemistry 267: 252-259; Engen and Smith (2001) Anal. Chem. 73: 256A-265A. Modification-Assisted Profiling (MAP), also known as Antigen Structure-based Antibody Profiling (ASAP) may be used to sort Abs binding the same antigen into groups of Abs binding different epitopes. MAP is a method that categorizes large numbers of Abs directed against the same antigen according to the similarities of the binding profile of each antibody to chemically or enzymatically modified antigen surfaces (see US 2004/0101920). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics.

As used herein, the term “flavivirus” refers to viruses belonging to the genus Flavivirus of the family Flaviviridae. According to virus taxonomy, about 50 viruses including ZIKV, DENV, YFV, JEV, WNV, and related flaviviruses are members of this genus. The viruses belonging to the genus Flavivirus are referred to herein as flaviviruses. Currently, these viruses are predominantly in East, Southeast and South Asia and Africa, although they may be found in other parts of the world.

As used herein, the term “Zika virus (ZIKV)” refers to a flavivirus which has been linked to microcephaly and other developmental abnormalities in the fetuses of pregnant women exposed to the virus (Schuler-Faccini et al., MMWR Morb. Mortal. Wkly. Rep. 2016, 65:59-62) as well as Guillian-Barre syndrome in adults (Cao-Lormeau et al., Lancet 2016, 387(10027):1531-9). The ZIKV may be from African or Asian genotype. A ZIKV possesses a positive sense, single-stranded RNA genome encoding both structural and nonstructural polypeptides. The genome also contains non-coding sequences at both the 5′- and 3′-terminal regions that play a role in virus replication. Structural polypeptides encoded by these viruses include, without limitation, capsid (C), precursor membrane (prM), membrane (M), and envelope (E) protein. Non-structural (NS) polypeptides encoded by these viruses include, without limitation, NS1, NS2A, NS2B, NS3, NS4A, NS4B, and NS5. The term “ZIKV” includes strains of ZIKV isolated from different ZIKV isolates, including ZikaSPH (Brazil 2015, GenBank accession No. KU321639.1), Brazil-ZKV (Brazil 2015, GenBank accession No. KU497555.1), PRVABC59 (Puerto Rico 2015, GenBank accession No. KU501215.1), Haiti1225 (Haiti 2014, GenBank accession No. KU509998.1), Natal RGN (Brazil, GenBank accession No. KU527068.1), SV0127-14 (Thailand 2014, GenBank accession No. KU681081.3), SPH2015 (GenBank accession No. KU321639.1), CPC-0740 (Philippine 2012, GenBank accession No. KU681082.3), SSABR1 (Brazil, GenBank accession No. KU707826.1), VE_Ganxian (China, GenBank accession No. KU744693.1), MR766-NIID (Uganda, GenBank accession No. LC002520.1), MR 766 (Uganda 1947, GenBank accession No. AY632535.2), and H/PF (French Polynesia 2013, GenBank accession No KJ776791.1) (WO 2017/109225). Further, ZIKV strains include Cambodia 2010 (GenBank accession No JN860885) or Micronesia 2007 (GenBank accession No EU545988) (Mlakar et al., N Engl J Med. 2016 Mar 10;374(10):951-8). Further, ZIKV strains include FLR (Colombia 2015) strain (WO 2018/017497), Z1106031 isolated in Suriname (Asian genotype; GenBank accession No KU312314), Z1106027 isolated in Suriname (Asian genotype; GenBank accession No KU312315); Z1106032 isolated in Suriname (Asian genotype; GenBank accession No KU312313), and Z1106033 isolated in Suriname (Asian genotype; Enfissi et al., Lancet 2016, 387(10015):227-228; GenBank Accession No. KU312312.1).

As used herein, the term “Dengue virus (DENV)” refers to a flavivirus possessing a positive sense, single-stranded RNA genome encoding both structural and nonstructural polypeptides. The genome also contains non-coding sequences at both the 5′- and 3′-terminal regions that play a role in virus replication. Structural polypeptides encoded by these viruses include, without limitation, capsid (C), precursor membrane (prM), membrane (M), and envelope (E) protein. Non-structural (NS) polypeptides encoded by these viruses include, without limitation, NS1, NS2A, NS2B, NS3, NS4A, NS4B, and NS5. DENV can be divided in different dengue serotypes. The term “DENV” may refer to DENV including all dengue serotypes.

The term “dengue serotype” as used herein, refers to a species of dengue virus which is defined by its cell surface antigens and therefore can be distinguished by serological methods known in the art. Four serotypes of dengue virus are known, i.e. dengue serotype 1 (DENV1), dengue serotype 2 (DENV2), dengue serotype 3 (DENV3), dengue serotype 4 (DENV4). The term “dengue serotype” includes strains of DENV isolated from different DENV isolates, for instance DENV1 strain Puerto Rico/US/BID-V853/1998 (GenBank accession No. EU482592.1), DENV2 strain Thailand/16681/84 (EMBL-EBI accession No: U87411.1), DENV3 strain Sri Lanka D3/H/IMTSSA-SRI/2000/1266 (GenBank accession No. AY099336.1), and DENV4 strain Dominica/814669/1981 (EMBL-EBI accession No: AF326825.1).

As used herein, the term “structural protein” refers to viral proteins that are structural components of the mature virus. Structural proteins include without limitation C, E, prM, and M proteins of a flavivirus. The term “structural proteins” may refer to at least one of the proteins including without limitation C, E, prM, and M protein of a flavivirus. The term “structural proteins” may also refer to all of the proteins including without limitation C, E, prM, and M protein of a flavivirus. The flavivirus may be a DENV or a ZIKV.

As used herein, the term “norovirus” refers to non-enveloped viruses comprising a single-stranded positive sense RNA. The viruses belong to the genus of Norovirus and the family of Caliciviridae. Noroviruses are transmitted directly from host to host and indirectly by contaminated water and food. Norovirus infection is characterized by nausea, vomiting, watery diarrhea, abdominal pain, and in some cases, loss of taste. A person usually develops symptoms of gastroenteritis 12 to 48 hours after being exposed to norovirus.

As used herein, the term “poliovirus” refers to non-enveloped viruses comprising a single-stranded positive sense RNA. The viruses belong to the genus of Enterovirus and the family of Picomaviridae. Infection occurs by the fecal—oral route and can cause poliomyelitis, which can result in inability to move by muscle weakness. Poliomyelitis can also be accompanied by minor symptoms such as fever and a sore throat, headache, neck stiffness and pains in the arms and legs.

As used herein, the term “live virus” refers to an infectious virus.

As used herein, the term “inactivated virus” or “live inactivated virus” refer to a live virus that has been inactivated, i.e. treated to lose its disease causing capacity. Inactivation can be carried out by various methods known in the art such as heat-inactivation, detergent-based inactivation, ultraviolet (UV) irradiation, gamma-irradiation, beta-propiolactone inactivation, or formaldehyde-based inactivation. An inactivated virus can be additionally purified by methods known in the art such as filtration or chromatography. The inactivated virus may be an inactivated zika virus. Consequently, when the vaccine antigen is an inactivated virus antigen, the vaccine can be referred to as “inactivated vaccine” or “live inactivated vaccine”. Consequently, when the vaccine antigen is a purified inactivated virus antigen, the vaccine can be referred to as “purified inactivated vaccine” or “purified live inactivated vaccine”. Consequently, when the vaccine antigen is a purified inactivated zika virus antigen, the vaccine can be referred to as “purified inactivated zika vaccine”. As inactivated vaccines induce a weaker immune response compared to live vaccines, immunological adjuvants and multiple “booster” injections may be required.

As used herein, the terms “immunological adjuvant” or “adjuvant” refer to substances that potentiate and/or modulate the immune response to an antigen to improve this response upon vaccination. Immunological adjuvants include inorganic adjuvants such as alum or organic adjuvants such as Freund's adjuvant.

As used herein the term “alum”, refers to an inorganic adjuvant including aluminum phosphate and aluminum hydroxide.

As used herein, the term “live attenuated virus” or “attenuated virus” refers to a live virus that has been attenuated (or weakened) from the germ that causes a disease in a host. A strategy for preparation of an attenuated virus is to populate the live virus in a foreign host. Upon population, the virus will accumulate mutations enabling the virus to grow well in the new host. The result is a virus population that is significantly different from the initial virus population. The goal is then to select a resulting virus population that is no longer harmful to the original host (which may be a human) and therefore is “attenuated”. Consequently, when the vaccine antigen is a live attenuated virus, the vaccine can be referred to as “live attenuated vaccine”, “live vaccine”, or “attenuated vaccine”.

As used herein, the term “vaccine” refers to a prophylactic material providing at least one vaccine antigen capable of introducing an immune response in a subject. The vaccine antigen may be derived from any material that is suitable for vaccination. A vaccine can be prepared by formulating the vaccine antigen. For example, the vaccine antigen may be a virus antigen such as a norovirus antigen, a zika virus antigen, a dengue virus antigen, or a poliovirus antigen and the corresponding vaccines may be referred to as noro vaccines, zika vaccines, dengue vaccines, or polio vaccines, respectively. A vaccine can be a purified inactivated vaccine or a live attenuated vaccine, when the vaccine antigen is a purified inactivated virus or a live attenuated virus. A vaccine can also be VLP vaccine, when the vaccine antigen is a VLP.

As used herein, the term “purified inactivated Zika vaccine (PIZV)” refers to a ZIKV vaccine that comprises ZIKV particles that have been amplified in culture and then inactivated to lose disease producing capacity. In addition to the inactivation step the ZIKV vaccine is purified. The ZIKV vaccine may be derived from ZIKV strain PRVABC59.

As used herein, the term “dose of vaccine” refers to a certain amount of vaccine. The term “dose of vaccine” may refer to an amount of vaccine given by one administration to a subject or an amount of vaccine given by all administrations to a subject i.e. including booster administrations. The vaccine may be a ZIKV vaccine, a norovirus vaccine, a dengue virus vaccine, or a poliovirus vaccine.

As used herein, the term “acceptor microsphere” refers to a microsphere that is capable of binding or is bound to the constant region of an acceptor Ab and that is not capable of binding to a donor antibody. Further, the acceptor microsphere is capable to accept energy which is transferred in a proximity reaction. Further, the acceptor microsphere comprises one or more molecules that are able to accept energy which is transferred in a proximity reaction and to thereby produce a detectable signal.

As used herein, the term “donor microsphere” refers to a microsphere that is capable of binding or is bound to the constant region of a donor Ab and that is not capable of binding to an acceptor Ab. Further, the donor microsphere is capable to donate energy which is transferred in a proximity reaction. Further, the donor contains one or more molecules that are able to donate energy which is transferred in a proximity reaction. Such a molecule may be a photosensitizer.

As used herein, the term “proximity reaction” refers to a reaction capable of producing a detectable signal. The proximity reaction is characterized by a donating step, wherein one of the two reaction partners (“the donor”, e.g. a donor microsphere) donates energy, which is transferred and by an accepting step, wherein the other of the two reaction partners (“the acceptor”, e.g. an acceptor microsphere) accepts the energy, which is transferred and thereby produces a detectable signal. The proximity reaction thus provides a signal dependent on the proximity of the two reaction partners and is therefore a read-out for the existence of the reaction partners within a certain distance. The intensity of the signal decreases with increasing distance of the reaction partners. If no signal can be detected, the reaction partners are in sufficient distance that no proximity reaction occurs. The proximity reaction and the corresponding distance of reaction partners detected by said proximity reaction is selected to distinguish between donors and acceptors which are bound to the same antigen molecule or particle and those which are not bound to said antigen molecule or particle, i.e. are not bound to any antigen molecule or particle or to other antigen molecules or particles.

As used herein, the term “complex” refers to a complex, wherein the donor Ab is bound to one epitope of an antigen by the donor Ab variable region and to the donor microsphere by the donor Ab constant region and the acceptor Ab is bound to another epitope of the antigen by the acceptor Ab variable region and the acceptor microsphere by the acceptor Ab constant region. Formation of a complex may bring donor and acceptor microsphere within a certain distance e.g. within 200 nm. It shall be noted at this point, that if the context states, expressions such as Ab/antigen complex, protein/antibody complex, antibody complex may have other meanings than described for the term “complex” in this paragraph.

As used herein, the term “signal” refers to a measurable event. The measurable event can include, but is not limited to luminescence, photoluminescence, fluorescence, chemoluminescence, and phosphorescence. The signal may be measured by any suitable detection instrument. The signal may be produced in a proximity reaction.

As used herein, the term “detection system” refers to any system which is suitable for detecting a signal indicative for the presence and/or the amount of a proximity reaction and therefore for the potency of an antigen sample. Examples for suitable detection instruments include but are not limited to EnVision®, EnSpire™, EnSight®, or VICTOR® Nivo™ Multilabel Plate Readers from Perkin Elmer.

As used herein, the term “acceptor antibody” refers to an Ab that is capable of binding or is bound to an acceptor microsphere. Further, the acceptor Ab is not capable of binding to a donor microsphere. In one embodiment the acceptor Ab is a monoclonal Ab.

As used herein, the term “donor antibody” refers to an Ab that is capable of binding or is bound to a donor microsphere. Further, the donor Ab is not capable of binding to an acceptor microsphere. In one embodiment the donor Ab is biotinylated at the constant region of the donor Ab. In one embodiment the donor Ab is a monoclonal Ab.

As used herein, the term “EC₅₀ value” refers to an amount of an antigen required to achieve 50% maximal complex formation at saturation with a certain pair of donor Ab and acceptor Ab. The amount can be expressed as a concentration or a titer (e.g. ng/μL, or TCID₅₀)

As used herein, the term “formulating” refers to the preparation of final vaccines by for instance addition of further substances to the vaccine antigen. A formulation step may include the addition of an adjuvant. For instance, the vaccine antigen is adsorbed on alum upon formulating the final vaccine.

### BRIEF DESCRIPTION OF THE DRAWINGS

FIG. 1 Analysis of PIZV with ZAPA. PIZV was serially diluted and analyzed with ZAPA using different mAb pairs. mAb pair #1: acceptor Ab is anti-ZIKV #2 and donor Ab is anti-ZIKV #1, mAb pair #2: acceptor Ab is anti-ZIKV #2 and donor Ab is anti-ZIKV #3, and mAb pair #3: acceptor Ab is anti-ZIKV #2 and donor Ab is anti-ZIKV #4. Presented are ZAPA signal counts in relative fluorescence units (RFU) depending on the PIZV concentrations [ng/mL] for each mAb pair.

FIG. 2 Analysis of ZIKV strain PRVABC59 with ZAPA. ZIKV was serially diluted and analyzed with ZAPA using different mAb pairs. mAb pair #1: acceptor Ab is anti-ZIKV #2 and donor Ab is anti-ZIKV #1, mAb pair #2: acceptor Ab is anti-ZIKV #2 and donor Ab is anti-ZIKV #3, and mAb pair #3: acceptor Ab is anti-ZIKV #2 and donor Ab is anti-ZIKV #4. Presented are ZAPA signal counts in relative fluorescence units (RFU) depending on the ZIKV TCID₅₀ titers for each mAb pair.

FIG. 3 Forced degradation study of drug substance (DS). Different amounts of heat-treated DS were mixed with untreated DS and analyzed with ZAPA using mAb pair #1 (acceptor Ab is anti-ZIKV #2 and donor Ab is anti-ZIKV #1). ZAPA values in antigen units per mL (AU/mL) were calculated by interpolating the ZAPA signal counts to a reference material. Presented are ZAPA values in AU/mL depending on the percentage of untreated DS.

FIG. 4 Forced degradation study of drug substance (DS) adsorbed on alum in PIZV. Different amounts of heat-treated DS were mixed with untreated DS and adsorbed on alum for formulation of PIZV and analyzed with ZAPA using mAb pair #1 (acceptor Ab is anti-ZIKV #2 and donor Ab is anti-ZIKV #1). ZAPA values in antigen units per mL (AU/mL) were calculated by interpolating the ZAPA signal counts to a reference material. Presented are ZAPA values in AU/mL depending on the percentage of untreated DS within PIZV samples.

FIG. 5 Analysis of different drug substance (DS) batches with ZAPA using mAb pair #1 (acceptor Ab is anti-ZIKV #2 and donor Ab is anti-ZIKV #1). DS batch #1 was diluted to result in antigen doses of 0.001, 0.005, 0.01, 0.05, 0.1 μg and adsorbed on alum previous to analysis with ZAPA. ZAPA values in antigen units per 100 μL (AU/100 μL) were calculated by interpolating the ZAPA signal counts to a reference material. Presented are ZAPA values in AU/100 μL depending on the dose of DS batch #1.

FIG. 6 Analysis of different drug substance (DS) batches with ZAPA using mAb pair #1 (acceptor Ab is anti-ZIKV #2 and donor Ab is anti-ZIKV #1). DS batch #2 was diluted to result in antigen doses of 0.001, 0.005, 0.01, 0.05, 0.1 μg and adsorbed on alum previous to analysis with ZAPA. ZAPA values in antigen units per 100 μL (AU/100 μL) were calculated by interpolating the ZAPA signal counts to a reference material. Presented are ZAPA values in AU/100 μL depending on the dose of DS batch #2.

FIG. 7 Analysis of different drug substance (DS) batches with ZAPA using mAb pair #1 (acceptor Ab is anti-ZIKV #2 and donor Ab is anti-ZIKV #1). DS batch #3 was diluted to result in antigen doses of 0.001, 0.005, 0.01, 0.05, 0.1 μg and adsorbed on alum previous to analysis with ZAPA. ZAPA values in antigen units per 100 μL (AU/100 μL) were calculated by interpolating the ZAPA signal counts to a reference material. Presented are ZAPA values in AU/100 μL depending on the dose of DS batch #3.

FIG. 8 Analysis of different drug substance (DS) batches with ZAPA using mAb pair #1 (acceptor Ab is anti-ZIKV #2 and donor Ab is anti-ZIKV #1). DS batch #4 was diluted to result in antigen doses of 0.001, 0.005, 0.01, 0.05, 0.1 μg and adsorbed on alum previous to analysis with ZAPA. ZAPA values in antigen units per 100 μL (AU/100 μL) were calculated by interpolating the ZAPA signal counts to a reference material. Presented are ZAPA values in AU/100 μL depending on the dose of DS batch #4.

FIG. 9 Neutralizing Ab titers induced in mice immunized with 0.01 μg dose of different drug substance (DS) batches # 1 to 4 formulated to PIZV by the adsorbance on alum. Presented are mean logarithmized EC₅₀ values as determined by a reporter virus particle (RVP) assay dependent on the DS batches.

FIG. 10 Correlation of ZAPA values with neutralizing Ab titers induced in mice immunized with different doses of drug substance (DS) batches #1 to 4 formulated to PIZV. Different antigen doses of 0.001, 0.005, 0.01, 0.05, 0.1 μg of the different DS batches were adsorbed on alum to formulate PIZV. ZAPA values in antigen units per 100 μL (AU/100 μL) were calculated by interpolating the ZAPA signal counts using mAb pair #1 (acceptor Ab is anti-ZIKV #2 and donor Ab is anti-ZIKV #1) to a reference material. Presented are ZAPA values in AU/100 μL for each dose of all DS batches #1 to 4 dependent on the mean logarithmized EC₅₀ values as determined by a reporter virus particle (RVP) assay.

FIG. 11 Analysis of PIZV test samples #1 and 2 and reference material with ZAPA. Samples and reference were serially diluted and analyzed with ZAPA using mAb pair #1 (acceptor Ab is anti-ZIKV #2 and donor Ab is anti-ZIKV #1). Presented are ZAPA signal counts in relative fluorescence units (RFU) depending on the PIZV test sample and reference dilution (presented as 1/dilution).

### DETAILED DESCRIPTION

### Kit of Acceptor Antibody, Donor Antibody, Acceptor Microsphere, and Donor Microsphere

The present invention is directed to a kit comprising an acceptor kit and a donor kit, the acceptor kit comprising an amount of an acceptor microsphere and an amount of an acceptor antibody and the donor kit comprising an amount of a donor microsphere and an amount of a donor antibody, wherein
the acceptor microsphere is capable to accept energy which is transferred in a proximity reaction to produce a signal and is capable of binding or is bound to the constant region of the acceptor antibody and is not capable of binding to the donor antibody,
the acceptor antibody has a variable region which is capable of binding to one of the at least two epitopes of an antigen and a constant region which is capable of binding or is bound to said acceptor microsphere, wherein the acceptor antibody is not capable of binding to the donor microsphere,
the donor microsphere is capable to donate energy which is transferred in a proximity reaction to produce a signal by the acceptor microsphere and is capable of binding or is bound to the constant region of the donor antibody and is not capable of binding to the acceptor antibody, and
the donor antibody has a variable region which is capable of binding to the other of the at least two epitopes of the antigen and a constant region which is capable of binding to said donor microsphere, wherein the donor antibody is not capable of binding to the acceptor microsphere.

Other settings applying two Abs binding to two epitopes of an antigen include for instance the sandwich ELISA setting. Thereby one Ab is immobilized onto a plate, the antigen is applied to that plate and can be bound by the immobilized Ab. Afterwards, the second Ab is added and an enzyme-based detection is carried out. Although ELISA is a common method applied in the art, it has several disadvantages. These disadvantages include the risk of false results due to insufficient blocking, the risk that the activity of the enzyme used for detection (e.g. horseradish peroxidase) may be hampered by sample constituents, as well as time-consuming operation (multiple steps required including washing procedures). Moreover, the colorimetric readout of the ELISA often lacks sensitivity as enzyme amplification is required and therefore is prone to variability and errors in the amount of amplification.

The microsphere useful for the invention ranges in the size from about 10 to about 500 nm in diameter, more preferably from about 50 to about 400 nm, even more preferably from about 200 to about 300 nm, and most preferably the microsphere has a diameter of about 200 to about 250 nm. The microsphere may be magnetic.

The microsphere may be constructed of any material to which molecules like Abs may be attached. For example, acceptable materials for the construction of microspheres include but are not limited to polystyrene, polyacrylic acid, polyacrylonitrile, polyacrylamide, polyacrolein, polybutadiene, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, or combinations thereof.

The microsphere may comprise functional groups useful for attachment of molecules, such as the Abs of the present invention. Said functional groups may be, but are not limited to, carboxylates, esters, alcohols, carbamides, aldehydes, amines, sulfur oxides, nitrogen oxides, or halides. Molecules can be covalently coupled to the microspheres using chemical techniques described herein or in the prior art (see e.g. Bruckner, Springer Verlag 2010, Organic Mechanisms). For example, Abs can be coupled to the microsphere by a reductive amination. Therefore, an aldehyde on the surface of the microsphere reacts with an amine group within the molecule to result in an unstable imine which is further reduced to a stable amine using suitable reducing agents such as sodium cyanoborohydride (NaBH₃CN) or sodium borohydride (NaBH₄).

As amine-containing compounds other than those provided by the Ab that should be coupled to the microsphere may interfere with the reductive amination, amine containing compounds should be removed from the Ab solution with a suitable buffer exchange method. For instance, buffers containing amines (e.g. Tris(hydroxymethyl)aminomethan (Tris), glycine, bicine, tricine) should be avoided. For instance, suitable buffer include phosphate buffer saline (PBS), carbonate buffer, or sodium phosphate buffer. The pH for reductive amination may be about 8. As coupling efficiency can be reduced, Abs should be free of any protein or peptide-based stabilizer such as bovine serum albumin (BSA) or gelatin and the buffer should be free of glycerol.

The microsphere may comprise affinity groups for attachment of molecules, such as Abs of the present invention. Said affinity groups may be, but are not limited to, Ni²⁺(for immobilization of His-tagged molecules like His-tagged Abs), Protein A, Protein G, Protein L, anti-human IgG Ab, anti-rabbit IgG Ab, anti-mouse IgG Ab, anti-mouse IgM Ab, anti-rat IgG Ab, anti-sheep IgG Ab, anti-chicken IgY Ab, anti-goat IgG Ab, anti-FLAG Ab, streptavidin, avidin, and glutathione.

Microspheres may be one out of the list consisting of AlphaLISA® acceptor microspheres, AlphaScreen® acceptor microspheres, AlphaDonor microspheres as produced by Perkin Elmer (Waltham, US). In certain embodiments the acceptor microsphere is an AlphaLISA® acceptor microsphere.

The acceptor microsphere is capable to accept energy which is transferred in a proximity reaction and comprises one or more molecules that are able to accept energy which is transferred in a proximity reaction. In certain embodiments, the one or more molecules are fluorophores. Fluorophores include but are not limited to thioxene, anthracene, rubrene, and lanthanides like europium, europium chelates, or any derivatives thereof. The fluorophores are able to produce a detectable signal upon excitation, wherein the excitation is caused by accepting energy which is transferred in a proximity reaction.

The donor microsphere is capable to donate energy which is transferred in a proximity reaction and contains one or more molecules that are able to donate energy which is transferred in a proximity reaction. In certain embodiments, the one or more molecules are photosensitizers. Photosensitizers are molecules that produce a chemical change in another molecule in a photochemical process. In certain embodiments the photosensitizer is phthalocyanine. The donation of energy, which is transferred, can be induced by irradiation of the photosensitizer with a certain wavelength as part of a proximity reaction.

In one embodiment the donor microsphere is not capable of directly interacting with an acceptor microsphere and the acceptor microsphere is not capable of directly interacting with a donor microsphere. “Directly” within that context means, that the acceptor and donor microsphere do react with each other in another way than occurring during a proximity reaction. For instance, the functional groups of the donor and acceptor microspheres do chemically react with each other or the affinity groups of the donor and acceptor microsphere do non-covalently interact with each other. For instance, a protein A-coated donor microsphere is able to interact directly with an anti-human IgG Ab-coated acceptor microsphere. This interaction is resulting a false-positive signal and should therefore be avoided.

A proximity reaction is a reaction capable of producing a detectable signal. The proximity reaction is characterized by a donating step, wherein one of the two reaction partners (“the donor”, e.g. a donor microsphere) donates energy, which is transferred and by an accepting step, wherein the other of the two reaction partners (“the acceptor”, e.g. an acceptor microsphere) accepts the energy which is transferred and thereby produces a detectable signal.

In one embodiment, the proximity reaction is characterized by a donating step, wherein the donor microsphere donates energy which is transferred and by an accepting step, wherein the acceptor microsphere accepts the energy which is transferred and thereby produces a detectable signal. In one embodiment the first step of a proximity reaction comprises irradiation of the donor microsphere with a certain wavelength, thereby inducing a chemical change in another molecule by the photosensitizer. In one embodiment the donor microsphere contains phthalocyanine and is irradiated with a wavelength of about 680 nm. Excited phthalocyanine induces the production of singlet oxygen out of ambient oxygen near and/or at the surface of the donor microsphere. Further, the proximity reaction is characterized by the diffusion of singlet oxygen to the acceptor microsphere. In the next step of the proximity reaction energy is transferred from singlet oxygen to a fluorophore (as for instance rubrene, anthracene, a europium chelate, or thioxene) within the acceptor microsphere. The energy may be further transferred from the fluorophore to one or more other fluorophores until the energy is transferred to a final fluorophore which emits light (the signal). In certain embodiments, light at a wavelength from about 520 to 680 nm is emitted and can be detected between about 520 to 630 nm.

In one embodiment of the invention fluorophores within the acceptor microsphere include thioxene, anthracene, and rubrene. Thioxene is converted to a di-ketone derivative following its reaction with singlet oxygen. Energy is transferred from the di-ketone derivative of thioxene to anthracene by emission of light with a wavelength of about 340 to about 350 nm, which results in an excitation of anthracene. Excited anthracene transfers energy to rubrene as the final fluorophore by emission of light with a wavelength of about 450 to about 500 nm. Excited rubrene produces a signal in the form of emission of light with a wavelength of about 540 to about 680 nm (the signal) which can be detected between about 520 and about 620 nm. An example of acceptor microspheres comprising thioxene, anthracene, and rubrene are the AlphaScreen® acceptor microspheres as produced by Perkin Elmer (Waltham, US).

In another embodiment of the present invention fluorophores within the acceptor microsphere include thioxene and a europium chelate. Thioxene is converted to a di-ketone derivative following its reaction with singlet oxygen. Energy is transferred from the di-ketone derivative of thioxene by its emission of light with a wavelength from about 340 to about 350 nm to europium as the final fluorophore. Excited europium produces a signal in the form of emission of light with a wavelength from about 605 to about 625 nm (the signal) which can be detected between about 607 and about 623 nm. An example of acceptor microspheres comprising thioxene and europium chelate are the Alpha LISA® acceptor microspheres as produced by Perkin Elmer (Waltham, US).

A long excitation wavelength of about 680 nm combined with a shorter emission wavelength of about 520 to about 620 nm reduces interference from biological or other assay components and thereby ensures a low background signal.

The proximity reaction is dependent on the proximity of the two reaction partners and is thereby indicative for the proximity of two reaction partners.

In one embodiment the requirement of sufficient proximity can be realized by the requirement of the diffusion of singlet oxygen from donor to acceptor microsphere. Singlet oxygen has a lifetime of about 4 ps prior to falling back to ground state. Within that time, singlet oxygen is able to diffuse about 200 nm in solution. The diffusion of singlet oxygen as basis of a proximity reaction is well suitable for analyzing antigens which result in complexes where the distance between donor and acceptor microsphere is 200 nm or less, e.g. virus particles with a diameter not exceeding 150 nm such as zika virus particles with a diameter of about 50 nm.

In one embodiment of the invention the at least two epitopes of the antigen are the same epitopes, wherein acceptor and donor Ab are capable of binding to the same epitope and/or have the same variable region. An antigen with at least two same epitopes may be a virus carrying multiple copies of structural protein on its surface or a dimeric virus antigen (e.g. the dimeric E protein of ZIKV).

In another embodiment of the present invention, the at least two epitopes are different epitopes and the acceptor and donor antibody have different variable regions.

In another embodiment the donor and acceptor Ab do not cross-react with other antigens. For instance, if the antigen is a ZIKV antigen, the donor and acceptor Ab do not cross-react with DENV antigens.

In one embodiment of the present invention at least one of the donor and acceptor Abs neutralizes the virus antigen to which it binds when tested in a plaque reduction neutralization test or reporter virus particle test or microneutralization test or focus forming assay.

In one embodiment of the invention the donor and acceptor antibody each neutralize the virus antigen to which they bind when tested in a plaque reduction neutralization test or reporter virus particle test or microneutralization test or focus forming assay.

In one specific embodiment of the invention the antigen is a virus antigen, including virus antigens selected from the group consisting of zika virus antigen, dengue virus antigen, norovirus antigen, and poliovirus antigen. The virus antigen may be one or more of the structural proteins or one or more the non-structural proteins of the virus. The virus antigen may also be the whole virus.

In another specific embodiment the antigen is a virus antigen, wherein the virus antigen is selected from the group consisting of a live virus, an inactivated virus, a live attenuated virus, and a virus like particle. In certain embodiments the antigen is selected from the group of a live zika virus, an inactivated zika virus, a live attenuated zika virus, and a zika virus like particle. In certain embodiments the antigen is selected from the group of a live dengue virus, an inactivated dengue virus, a live attenuated dengue virus, and a dengue virus like particle. In another embodiment the antigen is selected from the group of a live poliovirus, an inactivated poliovirus, a live attenuated poliovirus, and a poliovirus like particle. In another embodiment the antigen is selected from the group of a live norovirus, an inactivated norovirus, a live attenuated norovirus, and a norovirus like particle.

In one embodiment the antigen is a vaccine antigen. In a specific embodiment the vaccine antigen is a virus antigen, wherein the virus antigen is selected from the group consisting of a live virus, an inactivated virus, a live attenuated virus, and a virus like particle. In certain embodiments the antigen is selected from the group of a live zika virus, an inactivated zika virus, a live attenuated zika virus, and a zika virus like particle. In one embodiment the antigen is selected from the group of a live dengue virus, an inactivated dengue virus, a live attenuated dengue virus, and a dengue virus like particle. In another embodiment the antigen is selected from the group of a live poliovirus, an inactivated poliovirus, a live attenuated poliovirus, and a poliovirus like particle. In another embodiment the antigen is selected from the group of a live norovirus, an inactivated norovirus, a live attenuated norovirus, and a norovirus like particle. Within that context a virus antigen further includes virus antigens selected from the group consisting of zika virus antigen, dengue virus antigen, norovirus antigen, and poliovirus antigen. The virus antigen may be one or more of the structural proteins or one or more of the non-structural proteins of the virus. The virus antigen may also be the whole virus.

In one embodiment the virus antigen is adsorbed to an adjuvant. In certain embodiments the adjuvant is alum. Alum within this context may refer to aluminum hydroxide or aluminum phosphate.

In a specific embodiment the virus antigen is an inactivated virus.

In one embodiment the virus antigen is an inactivated virus adsorbed to an adjuvant. In certain embodiments the adjuvant is alum. Alum within this context may refer to aluminum hydroxide or aluminum phosphate.

In one specific embodiment the virus antigen is an inactivated zika virus.

In a more specific embodiment the virus antigen is an inactivated zika virus adsorbed to an adjuvant. In certain embodiments the adjuvant is alum. Alum within this context may refer to aluminum hydroxide or aluminum phosphate.

According to one embodiment each of the acceptor antibody, the donor antibody, the acceptor microsphere, and the donor microsphere is in an unbound state.

According to one embodiment the acceptor microsphere is bound to the constant region of the acceptor antibody and/or the donor microsphere is bound to the constant region of the donor antibody.

According to one embodiment of the present invention the donor antibody is biotinylated and the donor microsphere is coated with streptavidin.

According to one embodiment of the present invention the acceptor antibody is covalently bound to the acceptor microsphere. In a specific embodiment the acceptor Ab is covalently bound to the acceptor microsphere by a reductive amination.

According to one specific embodiment the donor antibody is biotinylated and the donor microsphere is coated with streptavidin and the acceptor antibody is covalently bound to the acceptor microsphere.

### Kit of Zika Binding Acceptor Antibody, Zika Binding Donor Antibody, Acceptor Microsphere, and Donor Microsphere

The present invention is directed to a kit, comprising an acceptor kit and a donor kit, the acceptor kit comprising an amount of an acceptor microsphere and an amount of an acceptor antibody and the donor kit comprising an amount of a donor microsphere and an amount of a donor antibody, wherein
the acceptor microsphere is capable to accept energy which is transferred in a proximity reaction to produce a signal and is capable of binding or is bound to the constant region of the acceptor antibody and is not capable of binding to the donor antibody,
the acceptor antibody has a variable region which is capable of binding to one of the at least two epitopes of a zika virus antigen and a constant region which is capable of binding or is bound to said acceptor microsphere, wherein the acceptor antibody is not capable of binding to the donor microsphere,
the donor microsphere is capable to donate energy which is transferred in a proximity reaction to produce a signal by the acceptor bead and is capable of binding or is bound to the constant region of the donor antibody and is not capable of binding to the acceptor antibody, and
the donor antibody has a variable region which is capable of binding to the other of the at least two epitopes of the zika virus antigen and a constant region which is capable of binding to said donor microsphere, wherein the donor antibody is not capable of binding to the acceptor microsphere.

Concerning the kit, reference is made to the chapter above entitled “Kit of acceptor antibody, donor antibody, acceptor microsphere, and donor microsphere”.

In one embodiment the donor and the acceptor Abs do not cross-react with dengue antigens.

In another embodiment at least one of the donor and the acceptor Abs is a ZIKV neutralizing Ab as for instance determined in a plaque reduction neutralization test or reporter virus particle test or microneutralization test or focus forming assay.

In another embodiment the donor and the acceptor Abs both are ZIKV neutralizing Abs as for instance determined in a plaque reduction neutralization test or reporter virus particle test or microneutralization test or focus forming assay.

In one embodiment the donor and acceptor Abs provide an EC₅₀ value towards the zika virus antigen of less than 100 ng/mL, or less than 80 ng/mL, or less than 60 ng/mL, or less than 40 ng/mL, or less than 30 ng/mL. In a specific embodiment within that context the zika virus antigen is a PIZV.

In another embodiment of the invention the donor and acceptor Abs provide an EC₅₀ value towards the zika virus antigen of less than 5e7 TCID₅₀ titer, or less than 4e7 TCID₅₀ titer, or less than 3e7 TCID₅₀ titer. In a specific embodiment within that context the zika virus antigen is a zika live virus.

The EC₅₀ value can be determined by detecting the signal indicative for the potency of the ZIKV antigen as described by the methods in the chapter below (“Method for determining the potency of an antigen sample”) for a serial dilution of the ZIKV antigen. By plotting the detected signal against the ZIKV antigen amount (which can be for instance either a concentration in ng/mL or a titer) and fitting the data with a non-linear regression according to a dose-response curve, the EC₅₀ value can be calculated.

According to one embodiment of the present invention the donor and acceptor Abs bind to epitopes on ZIKV EDIII of the E protein encoded by SEQ ID NO: 1.

According to one embodiment the acceptor and donor antibody are antibody 1 and antibody 2 and have different variable regions. Antibody 1 and antibody 2 can be anti-ZIKV #1 and anti-ZIKV #2. Further, antibody 1 and antibody 2 can be anti-ZIKV #2 and anti-ZIKV #3. For further details and characterization of Abs reference is made to Example 1. Antibody 1 and antibody 2 may each be characterized by the sequence of the VH-CDR1 and/or VH-CDR2 and/or VH-CDR3 and/or VL-CDR1 and/or VL-CDR2 and/or VL-CDR3. Antibody 1 and antibody 2 may each alternatively or additionally be characterized by the sequence of the VH and/or VL and/or H and/or L. The sequence referred to may be an amino acid sequence or a nucleic acid sequence encoding the amino acid sequence. The sequences and critical amino acid residues for binding are provided in Table 1 and 2, respectively. Critical residues are those amino acids whose side chains make the highest energetic contribution to the Ab-epitope interaction and whose mutation gave the lowest binding reactivities (<10% of wild-type) by alanine scanning mutagenesis (Bogan and Thorn, J. Mol. Biol. 1998, 280, 1-9; Lo Conte et al., J. Mol. Biol. 1999, 285, 2177-2198).

TABLE 1
Sequence information for antibody 1 and antibody 2. Antibody
1 and antibody 2 are presented together with their species
origin, amino acid sequences, as well as corresponding
nucleotide sequences of heavy chain (H), heavy chain variable
region (VH), heavy chain complementary determining regions
1 to 3 (VH-CDR-1-3), light chain (L), light chain variable
region (VL), as well as light chain complementary determining
regions 1 to 3 (VL-CDR-1-3).

| | | | Amino acid | Nucleic acid |
|---|---|---|---|---|
| Species | | | sequence | sequence |
| Origin | mAb | mAb part | (SEQ ID No) | (SEQ ID No) |
| Rabbit | Anti-ZIKV #1 | H | 2 | 12 |
| | (IgG, Clone 242-3) | VH | 3 | 13 |
| | | VH-CDR1 | 4 | N/A |
| | | VH-CDR2 | 5 | N/A |
| | | VH-CDR3 | 6 | N/A |
| | | L | 7 | 14 |
| | | VL | 8 | 15 |
| | | VL-CDR1 | 9 | N/A |
| | | VL-CDR2 | 10 | N/A |
| | | VL-CDR3 | 11 | N/A |
| Rabbit | Anti-ZIKV #2 | H | 16 | 26 |
| | (IgG, Clone 306-2) | VH | 17 | 27 |
| | | VH-CDR1 | 18 | N/A |
| | | VH-CDR2 | 19 | N/A |
| | | VH-CDR3 | 20 | N/A |
| | | L | 21 | 28 |
| | | VL | 22 | 29 |
| | | VL-CDR1 | 23 | N/A |
| | | VL-CDR2 | 24 | N/A |
| | | VL-CDR3 | 25 | N/A |
| Mouse | Anti-ZIKV #3 | H | 30 | 40 |
| | (IgG2a kappa Clone | VH | 31 | N/A |
| | D1-4G2-4-15) | VH-CDR1 | 32 | N/A |
| | | VH-CDR2 | 33 | N/A |
| | | VH-CDR3 | 34 | N/A |
| | | L | 35 | 41 |
| | | VL | 36 | N/A |
| | | VL-CDR1 | 37 | N/A |
| | | VL-CDR2 | 38 | N/A |
| | | VL-CDR3 | 39 | N/A |

TABLE 2
Critical amino acid residues in one-code letter code
from the ZIKV E protein (SEQ ID NO: 1) important for
binding of Anti-ZIKV #1 and 2 mAbs as evaluated
by alanine scanning mutagenesis. T = Thr, E = Glu, H = His.

| mAb | Critical Residues | E Protein Domain |
|---|---|---|
| Anti-ZIKV #1 (Clone 242-3) | E370 | III |
| Anti-ZIKV #2 (Clone 306-2) | T397, H398 | III |

According to one embodiment of the present invention, the antibody 1 is the donor antibody and the antibody 2 is the acceptor antibody.

According to another embodiment of the present invention, the donor antibody is biotinylated and the donor microsphere is coated with streptavidin.

According to another embodiment of the present invention, the acceptor antibody is covalently bound to the acceptor microsphere.

According to a specific embodiment of the present invention the antibody 1 is the donor antibody and antibody 2 is the acceptor antibody, the donor antibody is biotinylated and the donor microsphere is coated with streptavidin, and the acceptor antibody is covalently bound to the acceptor microsphere.

### Method for Determining the Potency of an Antigen Sample

The invention is directed to a method for detecting a signal indicative for the potency of an antigen sample such as a vaccine antigen sample, wherein the antigen in the antigen sample provides at least two epitopes and the method comprises the steps of:
Step 1: providing a kit comprising an acceptor kit and a donor kit, the acceptor kit comprising an amount of an acceptor microsphere and an amount of an acceptor antibody and the donor kit comprising an amount of a donor microsphere and an amount of a donor antibody, wherein
   the acceptor microsphere is capable to accept energy which is transferred in a proximity reaction to produce a signal and is capable of binding or is bound to the constant region of the acceptor antibody and is not capable of binding to the donor antibody,
   the acceptor antibody has a variable region which is capable of binding to one of the at least two epitopes of the antigen and a constant region which is capable of binding or is bound to said acceptor microsphere, wherein the acceptor antibody is not capable of binding to the donor microsphere,
   the donor microsphere is capable to donate energy which is transferred in a proximity reaction to produce a signal by the acceptor microsphere and is capable of binding or is bound to the constant region of the donor antibody and is not capable of binding to the acceptor antibody, and
   the donor antibody has a variable region which is capable of binding to the other of the at least two epitopes of the antigen and a constant region which is capable of binding to said donor microsphere, wherein the donor antibody is not capable of binding to the acceptor microsphere,
Step 2: contacting the amount of said donor microsphere, the amount of said acceptor microsphere, the amount of said donor antibody and the amount of said acceptor antibody of step 1 with the sample to allow forming a complex of the antigen in the sample with the donor antibody bound to the donor microsphere and the acceptor antibody bound to the acceptor microsphere and the acceptor antibody bound to one of the at least two epitopes of the antigen and the donor antibody bound to the other of the at least two epitopes of the antigen,
Step 3: conducting a proximity reaction to produce a signal indicative for the potency of the antigen sample, and
Step 4: detecting the signal indicative for the potency of the antigen sample.

The invention is further directed to such a method for determining the amount of the antigen in the antigen sample indicative for the potency of the antigen sample by detecting the signal in accordance with the method as described above and further comprising the step of:
Step 5: determining the amount of the antigen in the antigen sample indicative for the potency of the antigen sample based on the detected signal.

The invention is further directed to such a method for determining the potency of the antigen sample such as a vaccine antigen sample by detecting the amount of the antigen in accordance with the method as described above and further comprising the step of:
Step 6: determining the potency of the antigen sample based on the amount of the antigen in the sample determined in step 5.

Concerning the kit, reference is made to the previous chapters entitled “Kit of acceptor antibody, donor antibody, acceptor microsphere, and donor microsphere” and “Kit of zika binding acceptor antibody, zika binding donor antibody, acceptor microsphere, and donor microsphere”.

According to one embodiment the antigen sample is a vaccine antigen sample.

According to one embodiment the vaccine antigen in the vaccine antigen sample is a virus antigen.

According to one embodiment the antigen sample is a virus antigen sample.

Concerning the virus antigen, reference is made to the previous chapters entitled “Kit of acceptor antibody, donor antibody, acceptor microsphere, and donor microsphere” and “Kit of zika binding acceptor antibody, zika binding donor antibody, acceptor microsphere, and donor microsphere”.

In one embodiment contacting the amount of said donor microsphere, the amount of said acceptor microsphere, the amount of said donor antibody and the amount of said acceptor antibody of step 1 with the sample to allow forming a complex of the antigen in the sample with the donor antibody bound to the donor microsphere and the acceptor antibody bound to the acceptor microsphere and the acceptor antibody bound to one of the at least two epitopes of the antigen and the donor antibody bound to the other of the at least two epitopes of the antigen in step 2 is carried out for about 14 to 28 hours.

The order of contacting the amount of said donor microsphere, the amount of said acceptor microsphere, the amount of said donor antibody and the amount of said acceptor antibody of step 1 with the sample to allow forming a complex of the antigen in the sample with the donor antibody bound to the donor microsphere and the acceptor antibody bound to the acceptor microsphere and the acceptor antibody bound to one of the at least two epitopes of the antigen and the donor antibody bound to the other of the at least two epitopes of the antigen in step 2 may vary.

In one embodiment the amount of donor Ab and the amount of acceptor Ab are contacted with the sample for a certain contacting time in a first step followed by contacting the amount of donor microsphere and the amount of acceptor microsphere with the amount of donor Ab, the amount of acceptor Ab, and the sample for a certain contacting time in a second step.

In another embodiment the acceptor microsphere is bound to the constant region of the acceptor Ab and the donor microsphere is bound to the constant region of the donor Ab and the amount of acceptor microsphere bound to the constant region of the acceptor Ab and the amount of donor microsphere bound to the constant region of the donor Ab are concomitantly contacted with the sample for a certain contacting time.

In another embodiment, the donor Ab is biotinylated, the donor microsphere is coated with streptavidin and the acceptor microsphere is bound to the constant region of the acceptor Ab and the amount of donor Ab, as well as the amount of acceptor microsphere bound to the constant region of the acceptor Ab are contacted with the sample for a certain contacting time in a first step followed by contacting the sample, the amount of donor Ab, and the amount of acceptor microsphere bound to the acceptor Ab with the amount of donor microsphere for a second contacting time in a second step. Contacting in the first step may be carried out for about 16 to about 24 hours and contacting in the second step may be carried out for about 2 hours.

The complex allowed to form in step 2 brings the donor microsphere and acceptor microsphere in sufficient proximity that a proximity reaction can occur. Consequently, if no complex is formed, the donor microsphere and acceptor microsphere do not react in a proximity reaction. Therefore, the signal produced in the proximity reaction in step 3 is proportional to the amount of formed complex and therefore to the amount of antigen in the antigen sample.

In one embodiment of the invention the signal produced in the proximity reaction in step 3 is generated by the final fluorophore within the acceptor microsphere. In this context the final fluorophores may be a europium chelate or rubrene. The signal is emission of light with a wavelength in the range of about 520 to about 680 nm, in particular of about 615 nm. The signal can be detected by any suitable detection instrument.

In one embodiment the detection instrument is capable of excitation at about 680 nm and reading the emission at about 520 to about 630 nm, in particular at about 615 nm. A laser or a light emitting diode (LED) may be used as the excitation source. Preferred detection instruments may include but are not limited to EnVision®, EnSpire™, EnSight™, or VICTOR® Nivo™ Multilabel Plate Readers from Perkin Elmer.

The signal produced in the proximity reaction in step 3 is proportional to the amount of formed complex and therefore proportional to the amount of antigen in the antigen sample. Therefore, determining the amount of the antigen in the antigen sample in step 5 can be carried out by comparing the signal indicative for the potency of the antigen sample with a standard curve. The standard curve may be a sigmoidal-shaped dose-response curve or a linear curve plotting different amounts of the type of antigen to be analyzed within the sample against the corresponding signal. The amount of antigen can be for instance expressed as a concentration or a titer.

As the potency i.e. the capability of an antigen to induce an immune response in a subject depends on the amount of antigen within an antigen sample, the amount of antigen is indicative for the potency of the antigen sample, and therefore the signal indicative for the amount of antigen is also indicative for the potency of an antigen sample.

The invention is further directed to such a method for determining the potency of an antigen sample in accordance with the method as described above, wherein step 6 comprises the steps of
Step 6.1: determining the potency of standardized samples of the antigen in human or non-human subjects by measuring the associated mean neutralizing antibody titers produced in said human or non-human subjects,
Step 6.2: determining the amount of the antigen with at least two epitopes in said standardized samples according to the method as described above,
Step 6.3: establishing a standard curve from the mean neutralizing antibody titers of step 6.1 and the amount of the antigen of step 6.2, and
Step 6.4: determining the potency of the antigen sample by comparing the amount of antigen in the antigen sample determined in step 5 with the standard curve.

According to one embodiment the standardized antigen samples are provided by a forced degradation study or different doses of the antigen.

According to one embodiment the non-human subjects in step 6.1 include mice, rats, cats, rabbits, primates, and non-human primates.

According to one embodiment the subjects in step 6.1 are mice.

Mean neutralizing Ab titers can be determined by methods well known in the art including a MNT, a PRNT, a RVP assay, or a FFA. According to one embodiment of the invention mean neutralizing Ab titers are determined by a RVP assay.

According to one embodiment the standard curve is generated by plotting the potency of the standardized antigen samples expressed as the mean neutralizing Ab titers against the determined amount of the antigen in the standardized samples.

The present invention is further directed to the method as described above, wherein the antigen sample is a zika antigen sample. The zika antigen may be an inactivated virus. In certain embodiments of the present invention the method for monitoring the potency of a ZIKV antigen sample is referred to as Zika Antigen Potency Assay (ZAPA).

### Method for Monitoring the Potency of a Vaccine Antigen During the Production Process

The present invention is further directed to a method of monitoring the potency of a vaccine antigen during the production process including purifying, inactivating and formulating of said vaccine antigen to form a final vaccine by measuring the potency of the vaccine antigen in accordance with the method as described above.

In one embodiment the vaccine antigen is a ZIKV antigen and the potency of the ZIKV antigen is monitored during the production process including purifying, inactivating and formulating of said ZIKV antigen to form a final ZIKV vaccine by measuring the potency of the ZIKV antigen in accordance with the ZAPA method as described above.

Purifying can be carried out by filtration and/ or chromatography.

### Method of Producing a Virus Vaccine

The present invention is directed to a method of producing a virus vaccine comprising the steps of:
Step A: preparing various batches of vaccine antigen,
Step B: determining the potency of the vaccine antigen of the various vaccine antigen batches produced in step A in accordance with the method as described above and selecting the vaccine antigen batches in conformity with a predetermined potency requirement,
Step C: preparing vaccine batches by formulating the vaccine antigen batches selected in step B into various batches of virus vaccine, and
Step D: determining the potency of the vaccine antigen in the vaccine batches of the various batches produced in step C in accordance with the method as described above and selecting the vaccine batches in conformity with the predetermined potency requirement.

Concerning the method for determining the potency of the vaccine antigen reference is made to the previous chapters entitled “Method for determining the potency of an antigen sample” and “Monitoring the potency of a vaccine antigen during the production process”.

In one embodiment of the present invention step A includes various sub-steps and step B is performed after each sub-step.

In certain embodiments of the present invention the sub-steps include purification (as for instance by chromatography or filtration) and inactivation (as for instance with formaldehyde, or ultraviolet irradiation, or gamma irradiation, or beta-propiolactone).

In a specific embodiment the sub-steps comprise inactivation of a live virus to an inactivated virus.

In a specific embodiment of the present invention the live virus is a zika virus and the inactivation is accomplished with formaldehyde, or ultraviolet irradiation, or gamma irradiation, or beta-propiolactone.

The invention is further directed to a method as described above wherein the vaccine antigen is a zika antigen.

### Vaccines Obtainable by the Method for Producing a Virus Vaccine

The invention is further directed to a vaccine obtainable by the method described above.

The invention is further directed to a zika antigen obtainable by the method described above.

### Alternative Donor and Acceptor Structures

Microspheres have been described above as one suitable structure for an acceptor and a donor capable of reacting in a proximity reaction. However, the invention also encompasses other embodiments wherein alternative donor and acceptor structures are applied.

One example for alternative acceptor and donor structures is a pair which is able to react by a Förster Resonance Energy Transfer (FRET). For instance, in one specific embodiment a pair of two light-sensitive molecules (chromophores) are reacting in the proximity reaction as donor and acceptor. The donor chromophore is excited and can transfer the energy from the excitation to an acceptor chromophore through non-radiative dipole-dipole coupling. The excited acceptor chromophore is then capable to produce a detectable signal (e.g. by the emission of light). The efficiency of the energy transfer is inversely proportional to the sixth power of the distance between donor and acceptor. For instance, one common FRET pair of chromophores is cyan fluorescent protein (CFP) and yellow fluorescent protein (YFP), both are color variants of green fluorescent protein (GFP).

Another example for alternative acceptor and donor structures is a pair which is able to react in a Bioluminescence Resonance Energy Transfer (BRET). This technique uses a bioluminescent enzyme (e.g. Renilla luciferase) as a donor to produce an initial photon emission compatible with a fluorophore as YFP as an acceptor. BRET does not require external illumination to initiate the energy transfer, which decreases possible background noise.

In these embodiments the donor antibody may be covalently bound to a donor structure (such as the donor chromophore or the bioluminescent enzyme) and the acceptor antibody may be covalently bound to an acceptor structure (such as the acceptor chromophore), wherein the donor structure is capable of transferring energy (such as excitation energy in the case of a donor chromophore) to the acceptor structure if both structures are sufficiently close to each other.

The present invention is therefore further directed to kits and methods as described above, wherein the microspheres are exchanged by alternative donor and acceptor structures.

### EXAMPLES

The following Examples are included to demonstrate certain aspects and embodiments of the invention as described in the claims. It should be appreciated by those of skill in the art, however, that the following description is illustrative only and should not be taken in any way as a restriction of the invention.

### Example 1: Biotinylation of Donor Ab and Coupling of Acceptor Ab to Acceptor Microspheres

mAbs applied in the Zika antigen potency assay (ZAPA) set-up are listed in Table 1 and 3.

TABLE 3
Anti-ZIKV #4 mAb applied in the ZAPA. Anti-ZIKV #4 is
presented together with its species origin and amino acid sequences
of heavy chain (H), heavy chain variable region (VH), heavy
chain complementary determining regions 1 to 3 (VH-CDR-1-3),
light chain (L), light chain variable region (VL), as well as
light chain complementary determining regions 1 to 3 (VL-CDR-1-3).

| | | | Amino acid |
|---|---|---|---|
| Species | | | sequence |
| Origin | mAb | mAb part | (SEQ ID No) |
| Human | Anti-ZIKV #4 | H | 42 |
| | (Clone EDE1-C10) | VH | 43 |
| | | VH-CDR1 | 44 |
| | | VH-CDR2 | 45 |
| | | VH-CDR3 | 46 |
| | | L | 47 |
| | | VL | 48 |
| | | VL-CDR1 | 49 |
| | | VL-CDR2 | 50 |
| | | VL-CDR3 | 51 |

Anti-ZIKV #1 and 2 mAbs were generated and characterized as described in co-pending application PCT/US2019/052189 (Takeda Ig Application). In brief, rabbits were immunized with purified inactivated Zika vaccine (PIZV) and ZIKV virus like particles (VLPs). Afterwards, the spleen was isolated for generation of hybridoma cells. Hybridoma supernatants were examined for reactivity towards ZIKV VLPs and E protein, as well as cross-reactivity towards inactivated DENV 1-4 by enzyme linked immunosorbent assay (ELISA). Therefore, hybridoma supernatants were screened against inactivated DENV1 (West Pacific 74, Microbix), DENV2 (16681; Microbix), DENV3 (CH53489, Microbix), DENV4 (TVP-360, Microbix), ZIKV E protein (Native Antigen), and ZIKV VLP (Native Antigen). DENV1, 3, and 4 were inactivated with gamma-irradiation and DENV2 with formalin by the manufacturer as a part of the production process. Both ZIKV E protein and ZIKV VLP were used as positive control antigens. In brief, antigens were coated onto Nunc Polysorp ELISA plates at 1 μg/mL in carbonate coating buffer (pH 9.4) at 4° C. overnight prior to use. Then, plates were washed with PBS containing 0.05% Tween-20 (PBS-T). A 5% non-fat dry milk blocking solution was added to the plates for a minimum of 1 hour at room temperature to reduce non-specific binding. Plates were washed and hybridoma supernatants were added to the plates. Plates were then incubated at 37° C. for 1 to 2 hours. Plates were again washed with PBS-T. Goat-derived anti-rabbit IgG (H+L) horseradish peroxidase conjugated secondary Ab (Jackson ImmunoResearch, Lot. No. L2416-X326F) was diluted 1:5,000 in 5% milk blocking solution and added to the plates. Plates were incubated 37° C. for 1.5 hours and then washed again with PBS-T. 3,3′, 5,5′-Tetramethylbenzidine substrate was added and incubation was carried out for 10 min at room temperature. The reaction was stopped with 1 N HCI and the plates were scanned for absorbance at 450 nm and 630 nm using an EnSpire reader (Perkin Elmer). Positive binding cut-off was set at 0.5 optical density reading. Both, anti-ZIKV #1 and 2 did not show binding to any of DENV1 to 4 verifying that both Abs are ZIKV-selective (Table 4). Moreover, hybridoma supernatants were screened for their neutralizing activity in a microneutralization test (MNT) as well as a reporter virus particle (RVP) assay. Anti ZIKV #1 showed strong neutralization activity, whereas anti-ZIKV #2 showed weak neutralization activity. Affinity of hybridoma supernatants towards ZIKV VLPs was determined by a Bio-layer interferometry (BLI) assay. In addition, epitope binning was examined using a competitive BLI assay, binding a primary mAb to the VLP, followed by cross-binding a secondary mAb. Binning experiments showed that Anti-ZIKV #1 and 2 bind to different regions within the antigen. Further, mAbs were sequenced (comp. Table 1). Finally, amino acid residues within the antigen critical for binding of mAbs were evaluated using an alanine scanning mutagenesis library. Critical residues are those amino acids whose side chains make the highest energetic contribution to the Ab-epitope interaction and whose mutation gave the lowest binding reactivity (<10% of wild-type; Bogan and Thorn, J. Mol. Biol. 1998, 280, 1-9; Lo Conte et al., J. Mol. Biol. 1999, 285, 2177-2198). Both mAbs were shown to bind to ZIKV EDIII (comp. Table 2). Anti-ZIKV #1 and 2 were stored in PBS, pH 7.4 at a final concentration in the range of 1.3 to 1.4 mg/mL.

TABLE 4
Reactivity of Anti-ZIKV #1 and 2 hybridoma supernatants against
ZIKV VLP, ZIKV E protein, and DENV1 to 4 examined by ELISA.
Presented are optical density (OD) values for each antigen and Ab.

| | ZIKV | ZIKV E | | | | |
|---|---|---|---|---|---|---|
| mAb | VLP | protein | DENV1 | DENV2 | DENV3 | DENV4 |
| Anti-ZIKV #1 | 0.97 | 0.33 | 0.04 | 0.034 | 0.04 | 0.038 |
| Anti-ZIKV #2 | 1.15 | 0.61 | 0.037 | 0.034 | 0.034 | 0.048 |

Anti-ZIKV #3 was originally generated as described previously using DENV-2 whole virus for immunization of mice (Gentry et al., Am J Trop Med Hyg 1982, 31(3): 548-555). The mAb binds the fusion loop at EDII and shows cross-reactivity with other flaviviruses like ZIKV (Aubry et al., Transfusion 2016, 56:33-40). Anti-ZIKV #4 was originally generated as described previously using DENV-2 whole protein for immunization. The mAb binds to E protein dimer epitope and shows cross-reactivity with ZIKV (Barba-Spaeth et al., Nature 2016; 536:48-53).

Anti-ZIKV #3 and #4 are commercially available from Wuxi AppTec. Anti-ZIKV #3 is additionally available from Absolute Antigen (Protein A purified, supplied in PBS, pH 7.4 with 0.02% Proclin-300 at 1 mg/mL, Cat. No. Ab00230.2.0). Wuxi expressed both Abs in Chinese hamster ovary (CHO) cells. The supernatant of the transfected cells was affinity purified using Protein G sepharose column (GE Healthcare) and analyzed with SDS-PAGE. Heavy chain (H) sequence of anti-ZIKV #3 (SEQ ID NO: 30 and 40) is deposited in GenBank under the accession codes AHX42424.1 (amino acid sequence) and KJ438785.1 (coding sequence and amino acid sequence), light chain (L) sequence of anti-ZIKV #3 (SEQ ID NO: 35 and 41) is deposited in GenBank under the accession codes AHX42423.1 (amino acid sequence) and KJ438784.1 (coding sequence and amino acid sequence). Anti-ZIKV #4 has been crystalized complexed with DENV2 E protein (PDB: 4UT9; Rouvinski et al., Nature 2015, 520(7545): 109-113) and ZIKV (PDB: 5H37; Zhang et al., Nat Commun 2016, 7, 13679).

mAbs serving as acceptor Abs were coupled to acceptor microspheres as described in the following. For conjugation, 25 mg of acceptor microspheres (0.25 mL of a 100 mg/mL stock, unconjugated AlphaLISA® acceptor microspheres, Perkin Elmer, Cat. No. 6772001-3) were mixed with 0.5 mg of acceptor Ab to result in a coupling ratio of 1:50 (mg protein : mg microspheres). Next, corresponding volumes of 10% Tween-20 to result in a 160-fold dilution, corresponding volumes of a 25 mg/mL solution of NaBH₃CN (prepared freshly in water; Sigma Aldrich, Cat. No. 152159) to result in a 20-fold dilution, and corresponding volumes of 0.13 M phosphate buffer pH 8.0 were added to obtain a final reaction volume of 1 mL. For example, 0.374 mL of mAb concentrated at 1.34 mg/mL were added to 0.25 mL of the 100 mg/mL microsphere stock. Afterwards, 0.445 mL of 0.13 M phosphate buffer pH 8.0 were added, followed by 6 μL of 10% Tween-20 and 50 μL of a 25 mg/mL solution of NaBH₃CN. The mixture was incubated for 18-19 hours at 37° C. under mild agitation (6-10 rpm). For blocking, 50 μL of a 65 mg/mL solution of carboxy-methoxylamine (CMO; Sigma Aldrich, Cat. No. C13408) prepared freshly in 0.8 M NaOH were added to the reaction resulting in a final concentration of 3.25 mg/mL CMO and incubation was carried out for 1 hour at 37° C. For purification, the tube was centrifuged for 40 min at 16,000×g and 4° C., supernatant was removed, and the microsphere pellet was resuspended in 5 mL 0.1 M Tris-HCl, pH 8.0. After centrifugation for 40 min at 16,000×g and 4° C., supernatant was removed. The washing step was repeated once. After centrifugation for 40 min at 16,000×g and 4° C., supernatant was removed and the microspheres were resuspended to 5 mg/mL in storage buffer (PBS, pH 7.4 with 0.05% Proclin-300). The conjugated acceptor microspheres were stored at 4° C. until further use.

mAbs serving as donor Abs were biotinylated as described in the following. N-hydroxysuccinimido-ChromaLink™ Biotin (NHS-ChromaLink™ Biotin 354S, 10 mg/mL stock concentration in dimethylformamide (DMF); SoluLink Inc., Cat. No. B1001-105, Lot. No. WOTL26127) was prepared freshly at 2 mg/mL in PBS, pH 7.4. NHS-ChromaLink™ Biotin 354S contains a chromophore (aryl hydrazine) with an absorbance maximum at 354 nm linked by a triethylenglycol (PEG3) linker to biotin. The succinimidyl ester functional group enables modification of lysines in aqueous buffers. Diluted NHS-ChromaLink™ Biotin was mixed with the donor Ab to result in a 30-fold molar excess of biotin over Ab, wherein the reaction concentration of the Ab was kept at 0.5 mg/mL. The reaction volume was adjusted with PBS, pH 7.4 previous to addition of NHS-ChromaLink™ Biotin. For instance, 937 μL of 1.26 mg/mL mAb were mixed with 1333.3 μL PBS, pH 7.4. Next, 89.7 μL of 2 mg/mL NHS-ChromaLink™ Biotin were added, resulting in a total volume of 2360 μL and 1.18 mg mAb (7.375 nmoles) and 0.179 mg NHS-ChromaLink™ Biotin (221.250 nmoles) in the reaction. Incubation was carried out for 2 hours at 21-23° C. Afterwards, free biotin was removed using a desalting column equilibrated with PBS, pH 7.4 (Zeba desalting columns, 5 mL, Pierce (Thermo Fisher Scientific), Cat. No. 89882). This step was repeated once with a second desalting column. For characterization, absorbance at 280 nm (A₂₈₀ₙₘ, referring to the protein amount) and at 354 nm (A₃₅₄ₙₘ, referring to the biotin amount) were determined, wherein the 280 nm value was corrected for the absorbance of the label at 280 nm as determined as 0.23×A₃₅₄ₙₘ. With the extinction coefficient at 280 nm (214,400 M⁻¹) and the molecular weight (160,000 g/mol) of the mAb the protein concentration was determined from the A₂₈₀ₙₘ value. Likewise, the concentration of biotin was determined from the A₃₅₄ₙₘ value using an extinction coefficient of biotin at 354 nm of 29,000 M⁻¹ and a molecular weight of 810.92 g/mol. The ratio of biotin per Ab was then determined by dividing the concentration of biotin by the concentration of Ab. A final biotinylated Ab concentration of 0.5 μM (80 μg/mL) was adjusted by dilution with stabilization buffer (PBS, pH 7.4, 0.1% Tween-20, 0.05% sodium azide). Biotinylated mAbs were stored at 4° C. until further use.

### Example 2: Evaluation of mAb Pairs for ZAPA

Next, different combinations of mAbs were evaluated for their performance in the ZAPA (Table 5, mAb pairs #1 to 3). Donor Abs were biotinylated and acceptor Abs were coupled to acceptor microspheres according to Example 1.

TABLE 5
Different donor and acceptor Ab combinations tested in the ZAPA.

| mAb pair | Acceptor Ab | Donor Ab |
|---|---|---|
| #1 | Anti-ZIKV #2 | Anti-ZIKV #1 |
| #2 | Anti-ZIKV #2 | Anti-ZIKV #3 |
| #3 | Anti-ZIKV #2 | Anti-ZIKV #4 |

For testing of the mAb pairs, purified inactivated zika vaccine (PIZV) and ZIKV strain PRVABC59 were evaluated in ZAPA. PIZV was provided at a stock concentration of 10 μg/mL drug substance (DS; purified, liquid, formalin-inactivated ZIKV) as determined by a Bradford assay. PIZV is formulated by the absorbance of DS on aluminum hydroxide (Al(OH)₃; alum; Alhydrogel® 2%, Brenntag, Lot. No. 5414).

ZIKV TCID₅₀ (50% Tissue Culture Infectious Dose) was determined by an endpoint dilution assay including the observation of cytopathic effects (CPE) after inoculating Vero cells with the virus. Therefore, Vero cells were cultured in Dulbecco's Modified Eagle Medium (DMEM; Corning, Cat. No. 15-017-CV) supplemented with 10% (v/v) Fetal Bovine Serum (FBS; Sigma, Cat. No. 12007C), 2% (v/v) L-glutamine (from a 200 mM stock; Hyclone, Cat. No. SH30034.01), and 1% (v/v) Penicillin/Streptomycin (Pen/Strep, from a 10-fold stock; Hyclone, Cat. No. SV30010) at 36±2° C. and 5% CO₂. Cells were seeded at 1.4×10⁴ cells per well in 100 μL medium in a 96-well plate (Costar, Cat. No. 3596) and allowed to settle down and grow for 2 days to achieve a confluency of >90% at the time of virus addition. Then, cells were incubated with a serial dilution of ZIKV (prepared in dilution medium: DMEM supplemented with 2% (v/v) FBS, 2% (v/v) L-glutamine, and 1% (v/v) Pen/Strep) for 5 days±4 hours by decanting the supernatant from the cells and addition of 100 μL per well of corresponding virus dilution. The serial dilution was examined in duplicates. Each of the two equivalent dilution series was plated in quadruplicates. Negative controls were included by addition of 100 μL dilution medium lacking ZIKV. After the incubation time, the absorbance at 560 nm and 420 nm was recorded after incubating the plate for 15 min at room temperature to account for color changes in heavily infected wells in which the cells have died. Absorbance at 420 nm was subtracted from the absorbance at 560 nm. A value >0 accounted for CPE negative, a value <0 for CPE positive. From the CPE results for the dilutions and replicates, the mean TCID₅₀ titer per mL was calculated according to the method of Reed and Muench. The results were confirmed by scoring the plate visually with a light microscope and corrected if needed.

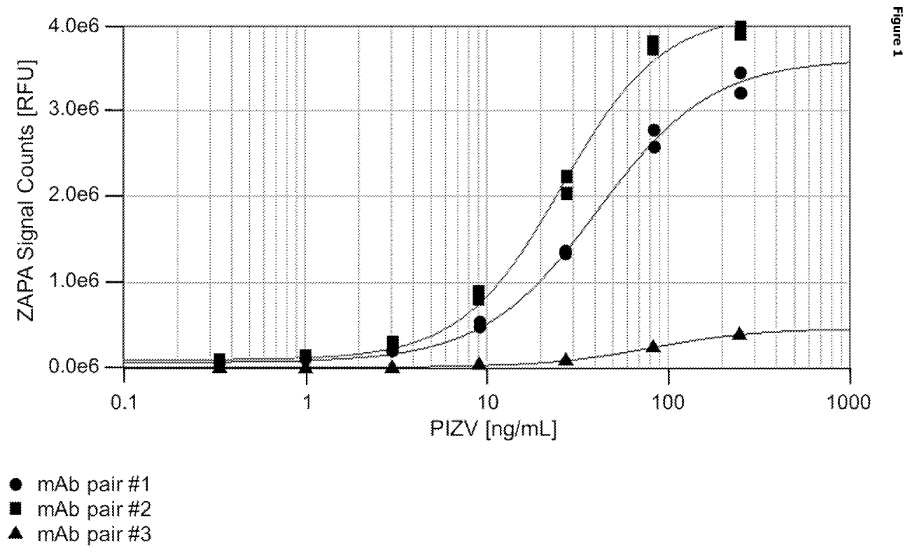

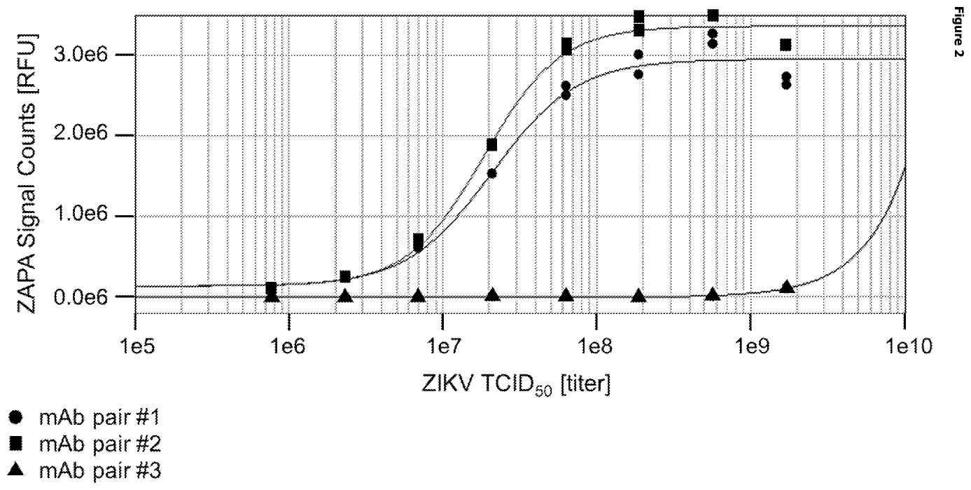

PIZV and ZIKV were serially diluted in assay buffer (25 mM HEPES pH 7.4, 0.5% Triton X-100, 0.1% Casein, 1 mg/mL Dextran-500, and 0.5% Proclin-300; prepared from a 10-fold stock from Perkin Elmer, Cat. No. AL000F) to result in a 5-fold amount of the final assay concentrations or titers, respectively, for each dilution (for final concentrations or titers see FIGS. 1 and 2). 10 μL per dilution were added per well into a white 96-well plate (1/2 area plate-96, Perkin Elmer, Cat. No. 6002299). In addition, blank wells were included by addition of 10 μL of assay buffer per well to account for background signal. Each dilution, as well as the blank control was evaluated in duplicates.

Biotinylated anti-ZIKV mAbs were prepared as described under Example 1 to result in a final concentration of 80 μg/mL biotinylated mAb. In a first step, the stock was vortexed for 5 to 20 sec and diluted 1:600 in assay buffer (e.g. 5 μL of biotinylated anti-ZIKV mAb to 2985 μL assay buffer). The dilution was again vortexed for 5 to 20 sec. Anti-ZIKV #2 was conjugated to acceptor microspheres as described under Example 1. In a second step, the 5 mg/mL stock of conjugated acceptor microspheres was vortexed (5 to 20 sec) and diluted 1:300 in assay buffer in the same tube as biotinylated anti-ZIKV mAbs were diluted (e.g. 10 μL of conjugated acceptor microspheres were added to 2990 μL diluted biotinylated mAb from the step before). The dilution was again vortexed for 5 to 20 sec. 30 μL of the dilution of biotinylated anti-ZIKV mAb and conjugated acceptor microspheres were added per well into the 96-well plate. The sides of the plate were tapped to collect contents to the bottom of the wells. The plate was sealed with a foil sealer (Adhesive PCR Sealing Foil Sheets, Thermo Fischer, Cat. No. AB-0626) to block light and incubation was carried out at 37° C. for 16 to 24 hours.

Streptavidin-coated donor microspheres at a 5 mg/mL stock concentration (PerkinElmer, Cat. No. 6760002) were vortexed (5 to 20 sec) and diluted 1:100 in assay buffer. 10 μL of the dilution were added per well. The sides of the plate were tapped to collect contents to the bottom of the wells. The plate was sealed with a foil sealer to block light and incubation was carried out at 37° C. for 2 hours ±10 min.

The plate was removed from the incubator and read within 10 min. Therefore, the foil sealer was removed from the plate immediately before reading to minimize light exposure. The plate was analyzed in an EnSpire multimode plate reader (PerkinElmer) with the “96-well AlphaLISA protocol”. ZAPA signal counts in relative fluorescence units (RFU) from the PIZV and ZIKV dilutions were normalized to the medium background signal resulting from the blank wells, and plotted against the corresponding PIZV concentrations and the ZIKV titers, respectively. The data were independently fitted for each mAb pair with a four parameter logistic (4PL) regression model (FIGS. 1 and 2).

mAb pairs #1 and 2 resulted in high signals for both, the ZIKV and PIZV samples. Contrarily, only weak signal for high PIZV concentrations and almost no signal even at the highest ZIKV titer was observed using mAb pair #3. The data show that the ZAPA set-up using mAb pairs #1 and 2 is able to efficiently determine PIZV and ZIKV in a concentration dependent manner, resulting in a good signal-to-noise ratio.

In summary, mAb pairs #1 and 2 resulted in high signals compared to mAb pair #3. ZAPA analysis was shown to fit for purpose of analyzing both, ZIKV strain PRVABC59 and PIZV. In conclusion, ZAPA mAb pairs #1 and 2 are able to efficiently measure the epitope availability from the live virus as well as from PIZV.

### Example 3: Evaluation of Stability Indication by ZAPA

In a next step, ZAPA was applied to analyze samples including different amounts of heat-inactivated DS, as well as PIZV samples formulated with the heat-inactivated DS samples by adsorbing DS on alum. The aim of this forced-degradation study was to evaluate if ZAPA is capable of reliably indicating the amount of intact DS, either present alone, or adsorbed on alum within the PIZV samples and therefore is a read out for antigen stability, i.e. intact epitopes.

Therefore, a portion of DS was heat treated for 1 h at 85° C. in order to degrade the material. DS samples were prepared by mixing untreated and heat-treated DS to result in 0, 25, 50, 75, and 100% of total heat-treated DS amount within the samples. Previous to preparing PIZV samples, DS samples were analyzed with a Bradford assay, using ZIKV recombinant E protein (Meridian Life Sciences, Inc.; Lot. No. 1J29317) as a standard. Of note, the total protein amount detected remained stable even after heat-treatment (Table 6).

TABLE 6
Analysis of DS samples with Bradford assay. Presented
is the total protein amount in μg/mL.

| DS Sample (% heat-inactivated DS) | Total protein amount (μg/mL) |
|---|---|
| 100 | 53.9 |
| 75 | 53.5 |
| 50 | 53.1 |
| 25 | 54.8 |
| 0 | 53.4 |

Next, DS samples were diluted to result in a DS amount of 1 μg per 100 μL volume (10 μg/mL). For formulation of PIZV samples, 40 μg of alum (Alhydrogel® 2%, Brenntag, Lot. No. 5414) were added per 1 μg DS sample and samples were stirred for 2 hours at room temperature. The DS and PIZV samples were stored at 5±3° C. until analysis.

DS and PIZV samples were analyzed with ZAPA using mAb pair #1 (see Table 5). In addition to DS and PIZV samples, a PIZV reference (stock concentration: 20 μg/mL) was included. The reference was serially diluted in assay buffer (25 mM HEPES pH 7.4, 0.5% Triton X-100, 0.1% Casein, 1 mg/mL Dextran-500, and 0.5% Proclin-300; prepared from a 10-fold stock from Perkin Elmer, Cat. No. AL000F) as described under Example 2.

10 μL per reference dilution or DS or PIZV sample were added per well into a white 96-well plate (1/2 area plate-96, Perkin Elmer, Cat. No. 6002299). In addition, blank wells were included by addition of 10 μL of assay buffer per well to account for background signal. Each reference dilution, as well as the samples and blank controls were evaluated in duplicates. ZAPA was further carried out as described under Example 2.

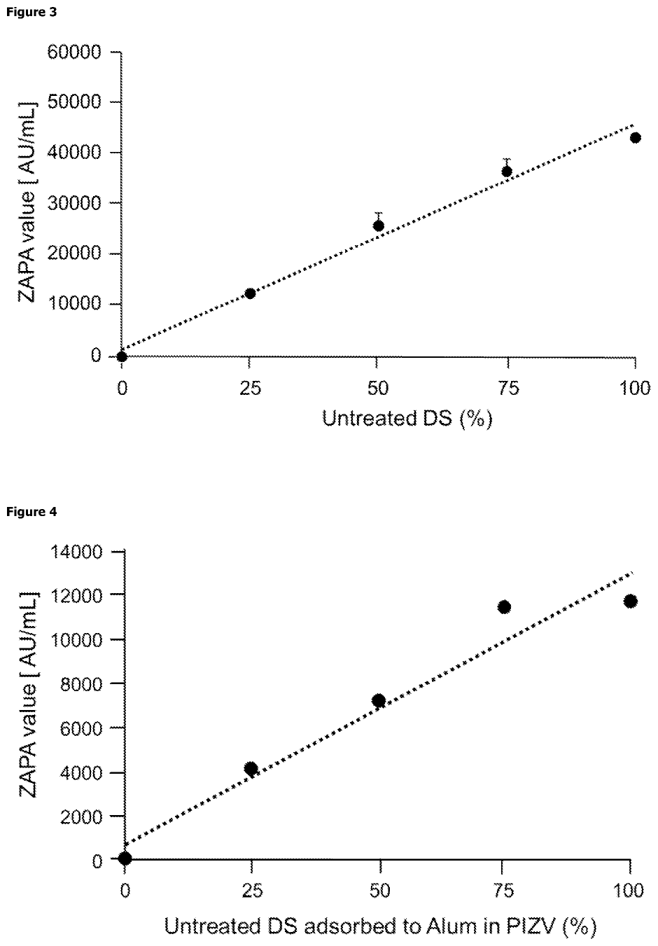

ZAPA signal counts in relative fluorescence units (RFU) from the reference dilutions, as well as from the DS and PIZV samples were normalized to the medium background signal resulting from the blank wells. The data from the reference material were independently fitted with a four parameter logistic (4PL) regression model. Corresponding ZAPA signal counts for a certain amount of intact DS within the PIZV reference dilutions were interpolated to the ZAPA signals from DS and PIZV samples and the thereby resulting ZAPA values were reported as antigen units per mL (AU/mL) for corresponding DS and PIZV samples (FIGS. 3 and 4).

DS and PIZV samples solely containing heat-treated DS resulted in the lowest ZAPA values compared to the other samples, whereas the DS and PIZV samples that contained 100% untreated DS resulted in the highest ZAPA values. The values of all examined DS and PIZV samples fit a linear response with an R² value of 0.986 (DS samples; linear regression: y=439.44x+1557.2) and 0.954 (PIZV samples; linear regression: y=122.14x+718.53), indicating that this method accurately and selectively detects changing antigen availability within the DS and PIZV samples after heat-degradation independent of the presence of additional ingredients such as alum.

It can be seen from the data that one or both of the epitopes responsible for binding of the mAbs used in ZAPA has or have been disrupted by heat treatment. The data indicate that ZAPA efficiently detects presence of intact epitopes within the samples upon heat-inactivation. In conclusion, the assay is sensitive to changes in sample stability.

Taken together, other than the Bradford assay which provides the total amount of protein independent of heat-degradation, ZAPA provides information about the amount of intact antigen. ZAPA shows robust performance and reliable evaluation of the amount of intact epitopes in the DS and PIZV samples, verifying that the method is stability indicating and not affected by the presence of additional ingredients such as alum.

### Example 4: Characterization of DS Batches Formulated to PIZV by ZAPA

In a next step, different DS batches formulated to PIZV were analyzed using ZAPA and compared to immune responses induced by the PIZV in CD-1 mice to evaluate if ZAPA is potency indicating.

Therefore, four different DS batches (#1 to 4) were analyzed. Total protein concentrations of DS batches were determined with Bradford as described under Example 3. Serial dilutions of the DS batches were prepared in Tris buffer (10 mM Hydroxymethyl aminomethane base (Fisher, Cat. No. T395-500) containing 150 mM sodium chloride (Fisher, Cat. No. S271-500), pH 7.6) to result in 0.001, 0.005, 0.01, 0.05, 0.1 μg of total antigen in 100 μL sample (reference is made to co-pending application U.S. 62/845,024). For formulation of PIZV samples, antigen was adsorbed on 50 μg alum (Alhydrogel® 2%, Brenntag, Lot. No. 5414) per 100 μL sample. The diluted samples were stored at 5±3° C. until analysis.

Next, PIZV samples from the different batches were analyzed with ZAPA using mAb pair #1 (see Table 5). In addition to PIZV samples, a PIZV reference (stock concentration: 20 μg/mL) was included. The reference was serially diluted in assay buffer (25 mM HEPES pH 7.4, 0.5% Triton X-100, 0.1% Casein, 1 mg/mL Dextran-500, and 0.5% Proclin-300; prepared from a 10-fold stock from Perkin Elmer, Cat. No. AL000F) as described under Example 2.

10 μL per reference dilution or PIZV sample were added per well into a white 96-well plate (1/2 area plate-96, Perkin Elmer, Cat. No. 6002299). In addition, blank wells were included by addition of 10 μL of assay buffer per well to account for background signal. Each reference dilution, as well as the samples and blank controls were evaluated in duplicates. ZAPA was further carried out as described under Example 2.

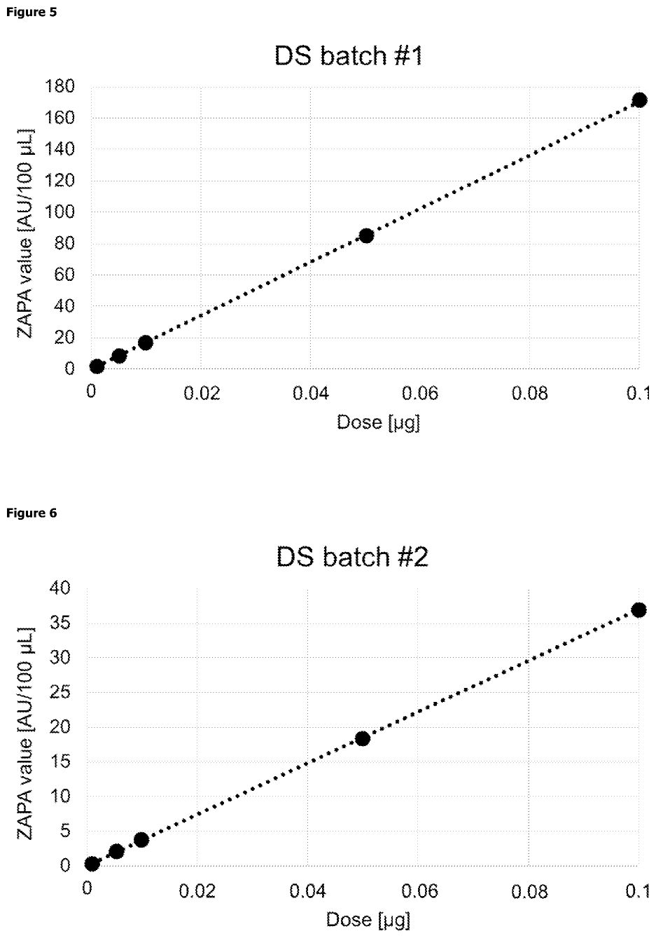

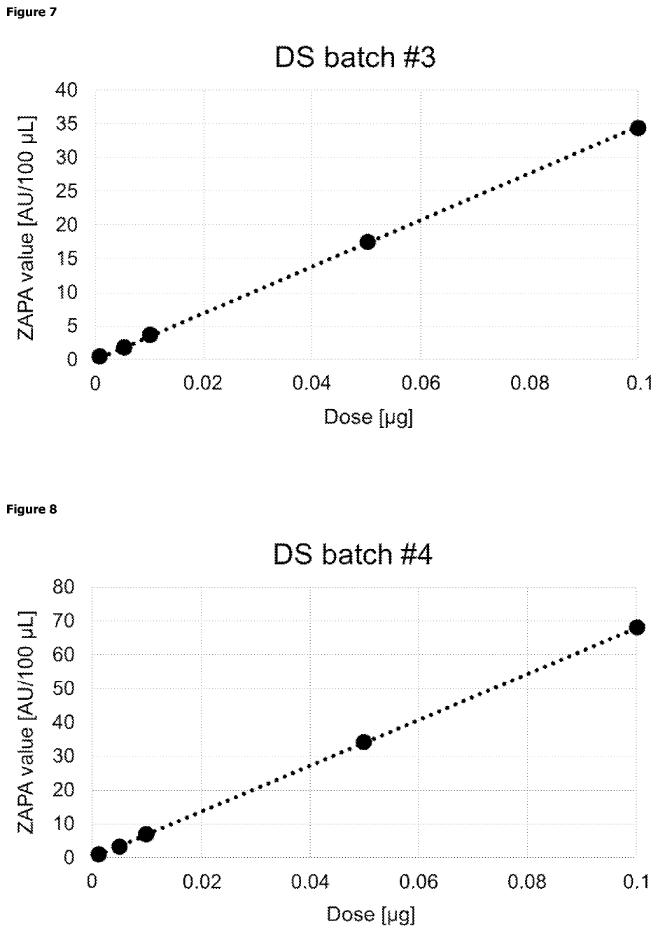

ZAPA signal counts in relative fluorescence units (RFU) from the reference dilutions, as well as from the PIZV samples were normalized to the medium background signal resulting from the two blank wells. The data from the reference material were independently fitted with a four parameter logistic (4PL) regression model. Corresponding ZAPA signal counts for a certain amount of intact DS within the PIZV reference dilutions were interpolated to the ZAPA signals from PIZV samples and thereby resulting ZAPA values were reported as antigen units per 100 μL (AU/100 μL) for the corresponding PIZV samples (FIG. 5-8).

ZAPA values for the dilution series of each DS batch followed a linear response, demonstrating robust performance of the assay as the ZAPA value linearly increases with epitope amount (FIG. 5-8). Although total protein amounts as calculated according to the Bradford analysis were the same for the corresponding dilutions (0.001-0.1 μg per 100 μL), ZAPA values differed amongst the DS batches indicating different amounts of intact epitopes within the samples. For instance, the samples containing 0.1 μg of total antigen per 100 μL resulted in ZAPA values of about 170, 37, 35, and 68 AU per 100 μL sample resulting from DS batches #1 to 4, respectively. This data indicates that the total protein amount as measured by a Bradford assay does not correlate with the intact epitopes within the sample in line with Example 3.

To link these in vitro ZAPA results indicative for antigenicity to immunogenicity and potency, CD-1 mice were vaccinated with corresponding PIZV samples resulting from the different DS batches. Therefore, for each of the PIZV samples eight mice including four male and four female mice were vaccinated by the intramuscular route with each one dose (volume of 100 μL) of PIZV sample. Neutralizing Ab titers after immunization were determined with a reporter virus particle (RVP) assay. Therefore, serum samples from mice, as well as a negative control lacking anti-ZIKV Abs (Innovative Research, Cat. No. IGRS-SER) and a positive control (Takeda) were heat-inactivated in a water bath at 56±2° C. for 30±2 min. After that, samples as well as negative and positive controls were serially diluted in assay media (1×Opti-MEM, Gibco, Cat. No. 11058-021, supplemented with 10% (v/v) FBS (Sigma, F4135) and 1% (v/v) Pen/Strep (100-fold stock, Gibco, 15140-122). 7.5 μL per dilution were added into one well of a white 384-well plate (Corning, Cat. No. 3570). ZIKV RVP particles (including C, E, prM, and M proteins; Integral Molecular) were diluted in assay media and 7.5 μL of the dilution were added per well into the 384-well plate. Incubation carried out for 60±2 min in a humidified incubator at 37±2° C. and 5% CO₂. Vero cells were cultured as described for the TCID₅₀ assay under Example 2. Cells were trypsinized, harvested, and resuspended in assay media prior to counting. 4625 cells in 15 μL assay media were added per well. Incubation carried out for 72±2 hours in a humidified incubator at 37±2° C. and 5% CO₂. Next, Renilla-Glo substrate (Promega, Cat. No. E2750) was diluted 1:100 in buffer according to the manufactures protocol. 30 μL of substrate dilution were added per well and incubation carried out for 15±2 min in the dark. Finally, the plate was analyzed with an Enspire reader (Perkin Elmer) and the half maximal effective concentration (EC₅₀) titer of neutralizing Abs is determined by regression of the recorded luminescence signal for the different dilutions.

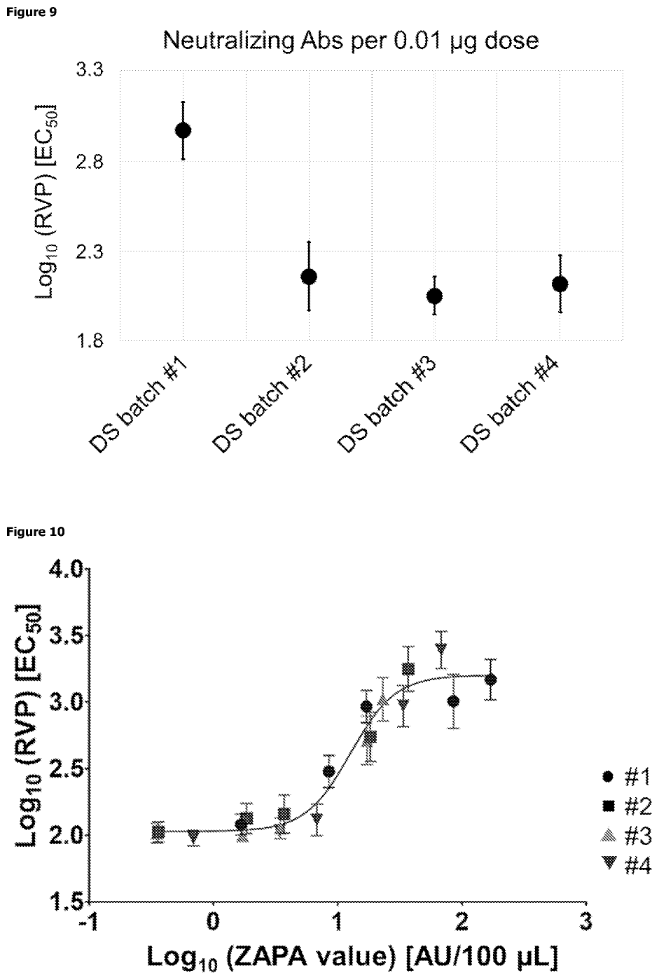

Of note, as already indicated by the different ZAPA values, neutralizing Ab titers differed for equal antigen doses depending on the DS batch. For instance, for a dose of 0.01 μg DS according to the Bradford assay, neutralizing Ab titers differed with a log₁₀ RVP value around 3 for DS batch #1 and log₁₀ RVP values around 2.1 for DS batches #2 to 4 (FIG. 9).

To examine whether ZAPA data correlate with the immunogenicity and potency results from the mouse model, data were analyzed using Prism (GraphPad, Version 8.2.0). Dose response curves (obtained by four parameter logistic (4PL) regression model) were compared using an F-test, examining two models. The first model (model 1) concludes that each agonist (meaning each PIZV dilution series) elicits the same dose response curve, whereas the second model (model 2) concludes that each agonist elicits a different dose response curve. The F ratio quantifies the relationship between the relative increase in the sum of squares from model 2 to model 1 and the relative increase in the degrees of freedom. If model 1 is correct, it is expected to measure an F ratio near 1.0. If the F ratio »1.0 there are two possibilities: model 2 is correct, or model 1 is correct, but random scatter led to a better fit using model 2. The p-value output qualifies how rare this ‘random scatter’ coincidence would be. In the case that the F ratio »1, and the p-value is low (less than α), it is concluded that model 2 is significantly better (more likely to be correct) than model 1. If the p-value is high, it is concluded that there is no compelling evidence supporting model 2 and model 1 is accepted.

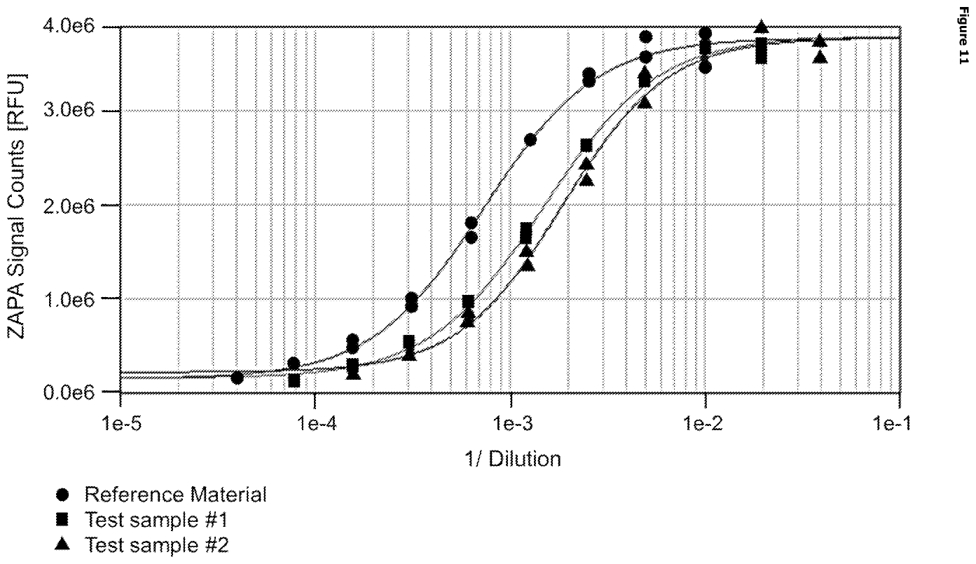

When comparing log₁₀-transformed AU-values (per 100 μL dose of sample) obtained by the ZAPA with the medium log₁₀-transformed RVP values within each diluted sample, model 1 is correct according to the F-test (F ratio=1.024, p-value 0.4301), meaning the same dose-response curve can be applied for each agonist (each PIZV dilution series; FIG. 10). According to this model, it can be concluded, that the ZAPA data correlated well with immune response in mice across the different DS batches applied.

Taken together, these data underline the benefit of the ZAPA for analyzing and characterizing different PIZV batches. Corresponding potency can be reliably predicted using the ZAPA, as assay results correlate well with immunogenicity of analyzed samples. In comparison, even if total antigen amounts as determined by Bradford are equal, epitopes and therefore potency of a sample can vary. ZAPA is a useful tool to account for such variations, as epitopes are reliably determined and the ZAPA signal is a direct indicator for antigenicity and in vivo immunogenicity, and therefore potency of the PIZV samples.

### Example 5: Determination of Relative Potency of PIZV Batches

ZAPA was shown to correlate well with antigenicity and in vivo immunogenicity and therefore potency of the PIZV samples under Example 4. Therefore, ZAPA can be applied to examine the relative potency of any PIZV batch compared to a PIZV reference of which ZAPA values have been correlated with induced neutralizing Ab titers (and therefore potency) as for example in a mouse as described under Example 4.

For this, two PIZV test samples (#1 and 2) and one PIZV reference (stock concentration: 20 μg/mL) were examined by ZAPA using mAb pair #1 (see Table 5). The samples and the reference were serially diluted in assay buffer (25 mM HEPES pH 7.4, 0.5% Triton X-100, 0.1% Casein, 1 mg/mL Dextran-500, and 0.5% Proclin-300; prepared from a 10-fold stock from Perkin Elmer, Cat. No. AL000F) as described under Example 2.

10 μL per reference dilution or PIZV test sample dilution were added per well into a white 96-well plate (1/2 area plate-96, Perkin Elmer, Cat. No. 6002299). In addition, blank wells were included by addition of 10 μL of assay buffer per well to account for background signal. Each dilution or sample, as well as blank was evaluated in duplicates. ZAPA was further carried out as described under Example 2.

ZAPA signal was analyzed by a four parameter logistic (4PL) regression independently for each dilution series as described under Example 2 (FIG. 11). To evaluate the relative potency of the two PIZV test samples a global four parameter logistic (4PL) fit with a parallel line analysis (PLA) was performed, specifying the reference material as the reference plot. Test sample #1 showed a relative potency of 0.500 with a standard error of 0.018 and an R² value for the global fit of 0.997, test sample #2 showed a relative potency of 0.408 with a standard error of 0.019 and an R² value for the global fit of 0.995.

In summary, ZAPA can be routinely applied to monitor different PIZV batches by evaluating the relative potency compared to a characterized reference in a fast, efficient, and reliable way.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Numerous references have been made to patents and printed publications throughout this specification. Each of the above-cited references and printed publications are individually incorporated herein by reference in their entirety.

With respect to the requirements within WIPO Standard ST.25 concerning the presentation of nucleotide and amino acid sequence listings in patent applications, the free text as used in the sequence listing is repeated in the following: “synthetic peptide”, “synthetic nucleotide”.

1. A method for detecting a signal indicative for the potency of an antigen sample such as a vaccine antigen sample, wherein the antigen in the antigen sample provides at least two epitopes and the method comprises the steps of:
Step 1: providing a kit comprising an acceptor kit and a donor kit, the acceptor kit comprising an amount of an acceptor microsphere and an amount of an acceptor antibody and the donor kit comprising an amount of a donor microsphere and an amount of a donor antibody, wherein
the acceptor microsphere is capable to accept energy which is transferred in a proximity reaction to produce a signal and is capable of binding or is bound to the constant region of the acceptor antibody and is not capable of binding to the donor antibody,
the acceptor antibody has a variable region which is capable of binding to one of the at least two epitopes of the antigen and a constant region which is capable of binding or is bound to said acceptor microsphere, wherein the acceptor antibody is not capable of binding to the donor microsphere,
the donor microsphere is capable to donate energy which is transferred in a proximity reaction to produce a signal by the acceptor microsphere and is capable of binding or is bound to the constant region of the donor antibody and is not capable of binding to the acceptor antibody, and
the donor antibody has a variable region which is capable of binding to the other of the at least two epitopes of the antigen and a constant region which is capable of binding to said donor microsphere, wherein the donor antibody is not capable of binding to the acceptor microsphere,
Step 2: contacting the amount of said donor microsphere, the amount of said acceptor microsphere, the amount of said donor antibody and the amount of said acceptor antibody of step 1 with the sample to allow forming a complex of the antigen in the sample with the donor antibody bound to the donor microsphere and the acceptor antibody bound to the acceptor microsphere and the acceptor antibody bound to one of the at least two epitopes of the antigen and the donor antibody bound to the other of the at least two epitopes of the antigen,
Step 3: conducting a proximity reaction to produce a signal indicative for the potency of the antigen sample, and
Step 4: detecting the signal indicative for the potency of the antigen sample.

2. A method for determining the amount of the antigen in the antigen sample indicative for the potency of the antigen sample by detecting the signal in accordance with claim 1 and further comprising the step of:
Step 5: determining the amount of the antigen in the antigen sample indicative for the potency of the antigen sample based on the detected signal.

3. A method for determining the potency of the antigen sample such as a vaccine antigen sample by detecting the amount of the antigen in accordance with claim 2 and further comprising the step of:
Step 6: determining the potency of the antigen sample based on the amount of the antigen in the sample determined in step 5.

4. The method for determining the potency of an antigen sample in accordance with claim 3, wherein step 6 comprises the steps of
Step 6.1: determining the potency of standardized samples of the antigen in human or non-human subjects by measuring the associated mean neutralizing antibody titers produced in said human or non-human subjects,
Step 6.2: determining the amount of the antigen with at least two epitopes in said standardized samples according to the method of claim 2,
Step 6.3: establishing a standard curve from the mean neutralizing antibody titers of step 6.1 and the amount of the antigen of step 6.2, and
Step 6.4: determining the potency of the antigen sample by comparing the amount of antigen in the antigen sample determined in step 5 with the standard curve.

5. The method of any one of claims 1 to 4, wherein the at least two epitopes are the same epitopes and wherein the acceptor and donor antibody have the same variable region and/or are capable of binding to the same epitope.

6. The method of any one of claims 1 to 4, wherein the at least two epitopes are different epitopes and wherein the acceptor and donor antibody have different variable regions.

7. The method of claims 1 to 6, wherein in step 1 the acceptor microsphere is bound to the constant region of the acceptor antibody and/or the donor microsphere is bound to the constant region of the donor antibody.

8. The method of any one of claims 1 to 7 wherein the antigen sample is a vaccine antigen sample.

9. The method of claim 8, wherein the vaccine antigen in the vaccine antigen sample is a virus antigen.

10. The method of any one of claims 1 to 7 wherein the antigen sample is a virus antigen sample.

11. The method of claim 9 or 10, wherein the donor and acceptor antibody do not cross-react with other virus antigens than the virus antigen of claims 9 and 10.

12. The method of any one of claims 9 to 11, wherein at least one or both of the donor and acceptor antibodies neutralize the virus antigen to which they bind when tested in a plaque reduction neutralization test or reporter virus particle test or microneutralization test or focus forming assay.

13. The method of any one of claims 9 to 12, wherein the virus antigen is selected from the group consisting of zika virus antigen, dengue virus antigen, norovirus antigen, and poliovirus antigen.

14. The method of any one of claims 9 to 12, wherein the virus antigen is selected from the group consisting of a live virus, an inactivated virus, a live attenuated virus and a virus like particle.

15. The method of claim 14, wherein the virus antigen is an inactivated virus.

16. The method of claim 15, wherein the virus antigen is an inactivated zika virus.

17. The method of claim 16, wherein the antigen is an inactivated zika virus absorbed on alum.

18. The method of any one of claims 4 to 17, wherein the standardized samples in step 6.1 are provided by a forced degradation study or different doses of the antigen.

19. The method of any one of claims 4 to 18, wherein the subjects in step 6.1 are mice.

20. A method of monitoring the potency of a vaccine antigen during the production process including purifying, inactivating and formulating of said vaccine antigen to form a final vaccine by measuring the potency of the vaccine antigen in accordance with a method of any one of claims 1 to 19.

21. A method of producing a virus vaccine comprising the steps of:
Step A: preparing various batches of vaccine antigen,
Step B: determining the potency of the vaccine antigen of the various vaccine antigen batches produced in step A in accordance with the method of claims 1 to 20 and selecting the vaccine antigen batches in conformity with a predetermined potency requirement,
Step C: preparing vaccine batches by formulating the vaccine antigen batches selected in step B into various batches of virus vaccine, and
Step D: determining the potency of the vaccine antigen in the vaccine batches of the various batches produced in step C in accordance with the method of claims 1 to 20 and selecting the vaccine batches in conformity with the predetermined potency requirement.

22. The method of claim 21, wherein step A includes various sub-steps and step B is performed after each sub-step.

23. The method of claim 22, wherein the sub-steps comprise inactivation of a live virus to an inactivated virus.

24. The method of claim 23, wherein the live virus is a zika virus and the inactivation is accomplished with formaldehyde, or ultraviolet irradiation, or gamma irradiation, or beta-propiolactone.

25. Vaccine obtainable by the method of claims 21 to 24.

26. A kit comprising an acceptor kit and a donor kit, the acceptor kit comprising an amount of an acceptor microsphere and an amount of an acceptor antibody and the donor kit comprising an amount of a donor microsphere and an amount of a donor antibody, wherein
the acceptor microsphere is capable to accept energy which is transferred in a proximity reaction to produce a signal and is capable of binding or is bound to the constant region of the acceptor antibody and is not capable of binding to the donor antibody,
the acceptor antibody has a variable region which is capable of binding to one of the at least two epitopes of a zika virus antigen and a constant region which is capable of binding or is bound to said acceptor microsphere, wherein the acceptor antibody is not capable of binding to the donor microsphere,
the donor microsphere is capable to donate energy which is transferred in a proximity reaction to produce a signal by the acceptor bead and is capable of binding or is bound to the constant region of the donor antibody and is not capable of binding to the acceptor antibody, and
the donor antibody has a variable region which is capable of binding to the other of the at least two epitopes of the zika virus antigen and a constant region which is capable of binding to said donor microsphere, wherein the donor antibody is not capable of binding to the acceptor microsphere.

27. The kit of claim 26, wherein the donor and acceptor antibodies do not cross-react with dengue antigens.

28. The kit of any one of claims 26 to 27, wherein at least one or both of the donor and acceptor antibodies are zika virus neutralizing antibodies.

29. The kit of any one of claims 26 to 28, wherein the donor and acceptor antibodies provide an EC₅₀ value towards the zika virus antigen of less than 100 ng/mL, or less than 80 ng/mL, or less than 60 ng/mL, or less than 40 ng/mL, or less than 30 ng/mL.

30. The kit of any one of claims 26 to 29, wherein the donor and acceptor antibodies bind to epitopes on the zika virus envelope glycoprotein domain III of the envelope glycoprotein encoded by SEQ ID NO: 1.

31. The kit of any one of claims 26 to 29, wherein the epitopes are two different epitopes and wherein the acceptor and donor antibody have different variable regions and wherein one of them is antibody 1 and the other is antibody 2.

32. The kit of claim 31, wherein antibody 1 binds to amino acid E370 of SEQ ID NO: 1 and antibody 2 binds to amino acids T397 and H398 of SEQ ID NO: 1.

33. The kit of claim 31, wherein antibody 1 and antibody 2 are each characterized by the heavy and light chain complementary determining regions, wherein
the antibody 1 is characterized by a heavy chain variable region (VH) comprising a heavy chain complementary determining region 1 (VH-CDR1) amino acid sequence of SEQ ID NO: 4, a heavy chain complementary determining region 2 (VH-CDR2) amino acid sequence of SEQ ID NO: 5, and a heavy chain complementary determining region 3 (VH-CDR3) amino acid sequence of SEQ ID NO: 6, and a light chain variable region (VL) comprising a light chain complementary determining region 1 (VL-CDR1) amino acid sequence of SEQ ID NO: 9, a light chain complementary determining region 2 (VL-CDR2) amino acid sequence of SEQ ID NO: 10, and a light chain complementary determining region 3 (VL-CDR3) amino acid sequence of SEQ ID NO: 11, and
the antibody 2 is characterized by a heavy chain variable region (VH) comprising a heavy chain complementary determining region 1 (VH-CDR1) amino acid sequence of SEQ ID NO: 18, a heavy chain complementary determining region 2 (VH-CDR2) amino acid sequence of SEQ ID NO: 19, and a heavy chain complementary determining region 3 (VH-CDR3) amino acid sequence of SEQ ID NO: 20, and a light chain variable region (VL) comprising a light chain complementary determining region 1 (VL-CDR1) amino acid sequence of SEQ ID NO: 23, a light chain complementary determining region 2 (VL-CDR2) amino acid sequence of SEQ ID NO: 24, and a light chain complementary determining region 3 (VL-CDR3) amino acid sequence of SEQ ID NO: 25.

34. The kit of claim 31, wherein antibody 1 and antibody 2 are characterized by the heavy and light chain complementary determining regions, wherein
the antibody 1 is characterized by a heavy chain variable region (VH) comprising a heavy chain complementary determining region 1 (VH-CDR1) amino acid sequence of SEQ ID NO: 32, a heavy chain complementary determining region 2 (VH-CDR2) amino acid sequence of SEQ ID NO: 33, and a heavy chain complementary determining region 3 (VH-CDR3) amino acid sequence of SEQ ID NO: 34, and a light chain variable region (VL) comprising a light chain complementary determining region 1 (VL-CDR1) amino acid sequence of SEQ ID NO: 37, a light chain complementary determining region 2 (VL-CDR2) amino acid sequence of SEQ ID NO: 38, and a light chain complementary determining region 3 (VL-CDR3) amino acid sequence of SEQ ID NO: 39, and
the antibody 2 is characterized by a heavy chain variable region (VH) comprising a heavy chain complementary determining region 1 (VH-CDR1) amino acid sequence of SEQ ID NO: 18, a heavy chain complementary determining region 2 (VH-CDR2) amino acid sequence of SEQ ID NO: 19, and a heavy chain complementary determining region 3 (VH-CDR3) amino acid sequence of SEQ ID NO: 20, and a light chain variable region (VL) comprising a light chain complementary determining region 1 (VL-CDR1) amino acid sequence of SEQ ID NO: 23, a light chain complementary determining region 2 (VL-CDR2) amino acid sequence of SEQ ID NO: 24, and a light chain complementary determining region 3 (VL-CDR3) amino acid sequence of SEQ ID NO: 25.

35. The kit of claim 31, wherein antibody 1 and antibody 2 are characterized by the heavy and light chain variable regions, wherein
the antibody 1 is characterized by a heavy chain variable region (VH) amino acid sequence of SEQ ID NO: 3 and a light chain variable region (VL) amino acid sequence of SEQ ID NO: 8, and the antibody 2 is characterized by a heavy chain variable region (VH) amino acid sequence of SEQ
ID NO: 17 and a light chain variable region (VL) amino acid sequence of SEQ ID NO: 22.

36. The kit of claim 31, wherein antibody 1 and antibody 2 are characterized by the heavy and light chain variable regions, wherein
the antibody 1 is characterized by a heavy chain variable region (VH) amino acid sequence of SEQ ID NO: 31 and a light chain variable region (VL) amino acid sequence of SEQ ID NO: 36, and
the antibody 2 is characterized by a heavy chain variable region (VH) amino acid sequence of SEQ ID NO: 17 and a light chain variable region (VL) amino acid sequence of SEQ ID NO: 22.

37. The kit of claim 31, wherein antibody 1 and antibody 2 are characterized by the heavy and light chain, wherein
the antibody 1 is characterized by a heavy chain (H) amino acid sequence of SEQ ID NO: 2 and a light chain (L) amino acid sequence of SEQ ID NO: 7, and
the antibody 2 is characterized by a heavy chain (H) amino acid sequence of SEQ ID NO: 16 and a light chain (L) amino acid sequence of SEQ ID NO: 21.

38. The kit of claim 31, wherein antibody 1 and antibody 2 are characterized by the heavy and light chain, wherein
the antibody 1 is characterized by a heavy chain (H) amino acid sequence of SEQ ID NO: 30 and a light chain (L) amino acid sequence of SEQ ID NO: 35, and
the antibody 2 is characterized by a heavy chain (H) amino acid sequence of SEQ ID NO: 16 and a light chain (L) amino acid sequence of SEQ ID NO: 21.

39. The kit of any one of claims 31 to 38, wherein antibody 1 is the donor antibody and antibody 2 is the acceptor antibody and wherein the donor antibody is biotinylated and the donor microsphere is coated with streptavidin and wherein the acceptor antibody is covalently bound to the acceptor microsphere.

40. Method of any one of claims 1 to 24, wherein the antigen is a zika antigen and the kit is defined by anyone of claims 26 to 39.

41. Antigen obtainable by a method of claim 40.
